(19) United States
(12) Patent Application Publication
(10) Pub. No.: US 2023/0235272 A1
(43) Pub. Date: Jul. 27, 2023

(54) Increased Lipid Production Through Metabolic Activation With Ionizing Radiation

(71) Applicants: University of Belgrade - Institute for Multidisciplinary Research, Belgrade (RS); Baylor University, Waco, TX (US); The University of Manchester, Manchester (GB); Varicon Aqua Solutions Ltd., Worcester (GB)

(72) Inventors: Ivan SPASOJEVIC, Belgrade (RS); Bernd ZECHMANN, Lorena, TX (US); Jon K. PITTMAN, Cheadle (GB); Alessandro Marco LIZZUL, London (GB); Marina STANIC, Belgrade (RS); Mima JEVTOVIC, Belgrade (RS); Milena DIMITRIJEVIC, Belgrade (RS); Jelena Danilovic LUKOVIC, Belgrade (RS); Snezana VOJVODIC, Belgrade (RS)

(73) Assignees: University of Belgrade - Institute for Multidisciplinary Research, Belgrade (RS); Baylor University, Waco, TX (US); The University of Manchester, Manchester (GB); Varicon Aqua Solutions Ltd., Worcester (GB)

(21) Appl. No.: 18/157,127

(22) Filed: Jan. 20, 2023

Related U.S. Application Data
(60) Provisional application No. 63/301,789, filed on Jan. 21, 2022.

(51) Int. Cl. C12N 1/12; C12M 1/34

(52) U.S. Cl. CPC C12N 1/12; C12M 41/38

(57) ABSTRACT

A method and system is provided for increasing lipids, biomass, and metabolite yields of a microalgae culture of cells and other organisms with conserved metabolic pathways compared to an untreated culture or organism when maintained under normal conditions. The method includes irradiating with electromagnetic ionizing radiation to induce rapid and reproducible hormetic metabolic activation in the organism cells. In an embodiment, the irradiation can be applied in a exponential or stationary phase of microalgae growth. The hormetic effect involves up-regulation of expression of lipid metabolism genes encoding enzymes that are involved in the biosynthesis of lipids with accumulation of energy reserves in the form of lipids and/or accumulation of other metabolites. The method can be implemented in a system that can interface with existing microalgae cultivation platforms, standard microalgae cultivation conditions, alongside standard microalgae culture types, and similarly with organisms with conserved metabolic pathways in their growth substrates.

### CROSS REFERENCE TO RELATED APPLICATIONS

This application claims the benefit of U.S. Provisional Application Serial No. 63/301,789, entitled “Increased Lipid Production In Microalgae Through Ionizing Radiation”, filed Jan. 21, 2022, which is incorporated herein by reference.

### STATEMENT REGARDING FEDERALLY SPONSORED RESEARCH OR DEVELOPMENT

Not applicable.

### REFERENCE TO APPENDIX

Not applicable.

### BACKGROUND OF THE INVENTION

### Field of the Invention

The disclosure relates generally to a novel hormesis process for increased performance of microalgae and organisms with conserved metabolic pathways. More specifically, the disclosure relates to a hormesis process for increased performance of microalgae and organisms with conserved metabolic pathways that increases lipid yield, biomass yield, and/or metabolite yield with hormetic beneficial doses of ionizing radiation.

### Description of the Related Art

Microalgae and many organisms with conserved metabolic pathways are utilized for commercial production of lipids, biomass, and metabolites that have applications for biofuels, feed, nutrition, cosmetic and pharmaceutical products, as well as a range of other products. Growth rates, lipid yield, and metabolites yield represent parameters with the strongest impact on the economic performance of farming of microalgae, and are also important within organisms with conserved metabolic pathways, and within related biotechnological fields. In many cases, these parameters have to be improved to reach commercial viability for bioproducts, biofuels and biomass production. Various processes have been used to increase performance of these parameters. However, it is common for existing processes to improve one of these parameters at the expense of the other. For example, existing cultivation processes for increased lipid productivity within microalgae often limit nutrients (typically nitrogen or phosphorus, “nutrient starvation”) to stress the cells into a lipid production mode, to the detriment of the overall growth rate. Such processes can require the use of phased production approaches, or the use of different cultivation media at various production stages, which increases the complexity and costs of the process. As a result of stress-induced negative effects on growth rate and biomass yield, an increase in lipid content may not result in higher lipid yield. High dose radiation has been used to develop new organisms as genetically mutant strains with increased lipid productivity. However, such mutants can be difficult and laborious to isolate and may possess other deleterious background mutations. High dose radiation has been used within studies to process biomass from microalgae and other sources to increase the content of simple sugars and other products. However, such processing is based on degradation of biomolecules and does not directly affect lipid or biomass yield.

There remains in the art the problem of increasing the yield of lipids, biomass, and metabolites in microalgae and different organisms with conserved metabolic pathways, without adversely affecting overall productivity.

### BRIEF SUMMARY OF THE INVENTION

The invention provides a method of increasing the performance of microalgae and organisms with conserved metabolic pathways compared to untreated organisms when maintained under normal conditions, and in particular the yield of lipids, biomass, and metabolites. The method comprises the use of electromagnetic ionizing radiation to induce rapid and reproducible metabolic activation via the hormetic effects of irradiation that is applied at specific absorbed doses and rates to organisms. In at least one embodiment, the irradiation is applied in the exponential or stationary phase of microalgae growth. The hormetic effect of electromagnetic ionizing radiation involves the up-regulation of expression of lipid metabolism genes encoding enzymes that are involved in the biosynthesis of lipids and accumulation of energy reserves in the form of lipids and/or the accumulation of other different metabolites and/or an increase in biomass. The method can be implemented using existing biotechnology cultivation platforms, including but not limiting to raceway ponds, photo-bioreactors, airlift bioreactors, bubble column bioreactors, fermentation bioreactors, biofilm reactors and other biofilm cultivation for microalgae and organisms grown on other cultivation/growth substrate, using standard cultivation/harvesting approaches, including but not limiting to, batch, semi-continuous, and continuous, under standard cultivation conditions, including but not limiting to photoautotrophic, heterotrophic and mixotrophic cultivation. Applicable organisms include, without limitation, classes of microalgae strains including freshwater and marine algae, such as without limitation, Bacillariophyta (diatoms), Chlorophyta (green algae), Cyanobacteria (blue-green algae), Haptophyta (mostly marine algae including coccoliths), Phaeophyta (brown algae), Pyrrophycophyta (dinoflagellates), Rhodophyta (red algae), and organisms with conserved metabolic pathways, such as without limitation, Bigyra (including Thraustochytrids), Streptophyta (plants including mosses), Ascomycota (fungi including yeasts), Basidiomycota (fungi including mushrooms), bacteria, and archaea. The present invention can interface with typical cells with at least one of X-ray and gamma radiation that produces hormesis in cells; and metabolic activation of the cells of such organisms to produce a hormetic effect in the culture under given conditions compared to an untreated culture of the same strain of such cells when maintained under the same conditions.

The disclosure provides a method of metabolic activation of organisms, comprising: irradiating the organisms with at least one of X-ray and gamma radiation that produces hormesis in organisms, and metabolically priming the organisms to produce a hormetic effect under given conditions compared to untreated organisms when maintained under the same conditions.

The disclosure provides a method of metabolic activation of an organism by irradiation, comprising: irradiating the organism with at least one of X-ray and gamma radiation that produces hormesis in the organism, the organism being at least one of a microalgae strain and an organism with conserved metabolic pathways; and metabolically priming the organism to produce a hormetic effect of at least one of a greater lipid yield, greater biomass yield, and greater metabolite yield under given conditions compared to untreated organisms of the same species when maintained under the same conditions

The disclosure also provides a system for increasing performance of a culture of at least one of microalgae and organisms with conserved metabolic pathways by irradiation, comprising: a cultivation platform comprising at least one of a raceway pond, a photo-bioreactor, an airlift bioreactor, a bubble column bioreactor, a fermentation bioreactor, and a biofilm reactor; an irradiation system with a source of at least one of X-ray and gamma radiation, coupled to the cultivation platform; and a controller configured to control activation of the source of radiation to irradiate the at least one of microalgae and organisms with conserved metabolic pathways at a predetermined phase of the culture for a period of time and rate of radiation and metabolically prime the at least one of microalgae and organisms with conserved metabolic pathways to produce a hormetic effect of at least one of a greater lipid yield, greater biomass yield, and greater metabolite yield of the culture under given conditions compared to an untreated culture of the same strain of the at least one of microalgae and organisms with conserved metabolic pathways when maintained under the same conditions.

The disclosure further provides a system for increasing performance of an organism with conserved metabolic pathways by irradiation, comprising: a growth substrate for the organism; an irradiation system with a source of at least one of X-ray or gamma radiation in operational proximity to the organism; and a controller configured to control activation the irradiation system to irradiate the organism for a period of time and rate of radiation and metabolically prime the organism to produce a hormetic effect of at least one of a greater lipid yield, biomass yield, and metabolite yield under given conditions compared to an untreated organism of the same species when maintained under the same conditions.

### BRIEF DESCRIPTION OF THE SEVERAL VIEWS OF THE DRAWINGS

FIG. 1 is a flowchart for an example of at least one embodiment using a method of the invention in a stationary phase of growth of microalgae and other single cell organisms with conserved metabolic pathways relevant to lipid yield.

FIG. 2A is a graph of an example of a growth curve of an organism exposed to the hormesis X-ray radiation of the invention with the process of FIG. 1.

FIG. 2B is a graph of an example of a statistically significant positive impact on lipid yield of the organism of FIG. 2A for given irradiation dosing at various dosing rates.

FIG. 2C is a graph of an example of no statistically significant effect versus control on biomass yield of the organism of FIG. 2A for given irradiation dosing at various dosing rates.

FIG. 2D is a graph of an example of a statistically significant positive impact on lipid droplets of cells of the organism of FIG. 2A for given irradiation dosing at various dosing rates.

FIG. 2E1 is micrograph of an untreated cell of the organism of FIG. 2A with limited number of lipid droplets.

FIG. 2E2 is a micrograph of a treated cell of the organism of FIG. 2A with a given irradiation dose and rate showing increased lipid droplets over the untreated cell of the organism of FIG. 2E1.

FIG. 2E3 is a micrograph of a treated cell of the organism of FIG. 2A with a given irradiation dose and rate showing increased lipid droplets over the untreated cell of the organism of FIG. 2E1.

FIG. 2E4 is a micrograph of a treated cell of the organism of FIG. 2A with a given irradiation dose and rate showing increased lipid droplets over the untreated cell of the organism of FIG. 2E1.

FIG. 2F is a graph of an example of no statistically significant effect versus control on a fatty acids profile of cells of the organism of FIG. 2A for given irradiation dosing at various dosing rates.

FIG. 2G is a graph of an example of a statistically significant positive impact on metabolite alpha-linoleic acid (omega-3 fatty acid) content in biomass of the organism of FIG. 2A for an exemplary irradiation dosing at a given dosing rate.

FIG. 2H is a graph of an example of a growth curve of another organism exposed to the X-ray radiation of the invention with the process of FIG. 1.

FIG. 2I is a graph of an example of a statistically significant positive impact on relative lipid yield of the organism of the FIG. 2H for given irradiation dosing at various dosing rates.

FIG. 2J is a graph of an example of no statistically significant effect versus control on biomass yield of the organism of the FIG. 2H for given irradiation dosing at various dosing rates.

FIG. 2K is a graph of an example of positive impact of irradiation of the invention on up-regulation of expression of different classes of genes, including lipid metabolism genes in the organism of FIG. 2A.

FIG. 2L is a graph of an example of up-regulation of expression of lipid metabolism genes for the production of fatty acids and triacylglycerols in lipid droplets in the organism of FIG. 2A.

FIG. 2M is a diagram explaining an exemplary of the process of metabolic activation and up-regulation of expression of lipid metabolism genes for the production of fatty acids and triacylglycerols in lipid droplets for the irradiation on a cell of an organism of FIG. 2A.

FIG. 2N is a diagram of an example of a phylogenetic tree of a lipid metabolism gene Acetyl-CoA carboxylase, biotin carboxylase (ACCase) for the synthesis of fatty acids in lipid droplets, including from the green algae organism of FIG. 2A and FIG. 2H, showing high evolutionary conservation across classes of microalgae and organisms with conserved metabolic pathways, including plants, yeast, fungi, bacteria and archaea.

FIG. 2O is a diagram of an example of a phylogenetic tree of a lipid metabolism gene Long-chain acyl-CoA synthetase (LACS) for the synthesis of fatty acids in lipid droplets, including from the green algae organism of FIG. 2A and FIG. 2H, showing high evolutionary conservation across classes of microalgae and organisms with conserved metabolic pathways, including plants, yeast, fungi, bacteria and archaea.

FIG. 2P is a diagram of an example of a phylogenetic tree of a lipid metabolism gene Lysophosphatidic acid acyltransferase (LPAAT) for the synthesis of triacylglycerols in lipid droplets, including from the green algae organism of FIG. 2A and FIG. 2H, showing high evolutionary conservation across classes of microalgae and organisms with conserved metabolic pathways, including plants, yeast, fungi, bacteria and archaea.

FIG. 2Q is a diagram of an example of a phylogenetic tree of a lipid metabolism gene Phosphatidic acid phosphatase (PAP) for the synthesis of triacylglycerols in lipid droplets, including from the green algae organism of FIG. 2A and FIG. 2H, showing high evolutionary conservation across classes of microalgae and organisms with conserved metabolic pathways, including plants, yeast, fungi, bacteria and archaea.

FIG. 2R is a diagram of an example of a phylogenetic tree of a lipid metabolism gene Diacylglycerol acyltransferase (DGAT) for the synthesis of triacylglycerols in lipid droplets, including from the green algae organism of FIG. 2A and FIG. 2H, showing high evolutionary conservation across classes of microalgae and organisms with conserved metabolic pathways, including plants, yeast, fungi, bacteria and archaea.

FIG. 3 is a flowchart for an example of at least one embodiment using a method of the invention in an exponential phase of growth of microalgae and other organisms with conserved metabolic pathways relevant to lipid yield.

FIG. 4A is a graph of an example of a growth curve of the organism of FIGS. 2A-2G, but exposed to the hormesis X-ray radiation of the invention with the process of FIG. 3.

FIG. 4B is a graph of an example of a positive impact on cell density of the organism of FIG. 4A for given irradiation dosing at various dosing rates.

FIG. 4C is a graph of a statistically significant positive impact on growth rate of the organism of FIG. 4A for given irradiation dosing at various dosing rates.

FIG. 4D is a graph of an example of a statistically significant positive impact on biomass yield of the organism of FIG. 4A for given irradiation dosing at various dosing rates.

FIG. 5A is a top view schematic diagram of an example of an irradiation system that is mounted on a raceway pond.

FIG. 5B is an enlarged top view schematic diagram of the irradiation system of FIG. 5A.

FIG. 5C is an enlarged side sectional view schematic diagram through a longitudinal section of the irradiation system of FIG. 5A.

FIG. 5D is an enlarged side sectional view schematic diagram through a lateral section of the irradiation system of FIG. 5A.

FIG. 5E is an enlarged side sectional view schematic diagram of irradiation beam coverage through a longitudinal section of the irradiation system of FIG. 5A.

FIG. 6A is a side view schematic diagram of another example of an irradiation system that is mounted on a photo-bioreactor.

FIG. 6B is an enlarged top view schematic diagram of the irradiation system of FIG. 6A.

FIG. 6C is an enlarged side sectional view schematic diagram through a longitudinal section of the irradiation system of FIG. 6A.

FIG. 6D is an enlarged side sectional view schematic diagram through a lateral section of the irradiation system of FIG. 6A.

FIG. 6E is an enlarged side sectional view schematic diagram of irradiation beam coverage through a longitudinal section of the irradiation system of FIG. 6A.

FIG. 7A is a side view schematic diagram of another example of an irradiation system that is mounted on an airlift bioreactor.

FIG. 7B is an enlarged top view schematic diagram of the irradiation system of FIG. 7A.

FIG. 7C is an enlarged side sectional view schematic diagram through a longitudinal section of the irradiation system of FIG. 7A.

FIG. 7D is an enlarged side sectional view schematic diagram of irradiation beam coverage through a longitudinal section of the irradiation system of FIG. 7A.

FIG. 8A is a side view schematic diagram of another example of an irradiation system that is mounted on a wall of a fermentation bioreactor.

FIG. 8B is an enlarged top view schematic diagram of the irradiation system of FIG. 8A.

FIG. 8C is an enlarged side sectional view schematic diagram through a longitudinal section of the irradiation system of FIG. 8A.

FIG. 8D is an enlarged side sectional view schematic diagram of irradiation beam coverage through a longitudinal section of the irradiation system of FIG. 8A.

FIG. 9A is a side view schematic diagram of another example of an irradiation system that is mounted on biofilm reactor.

FIG. 9B is an enlarged side sectional view schematic diagram through a longitudinal section of the irradiation system of FIG. 9A.

FIG. 10A is a side view schematic diagram of another example of an irradiation system with multipurpose mobility for the treatment of organisms with conserved metabolic pathways relevant to lipid yield.

FIG. 10B is an enlarged top view schematic diagram of the irradiation system of FIG. 10A.

FIG. 10C is an enlarged side sectional view schematic diagram through a longitudinal section of the irradiation system of FIG. 10A.

### DETAILED DESCRIPTION

The Figures described above and the written description of specific structures and functions below are not presented to limit the scope of what Applicants have invented or the scope of the appended claims. Rather, the Figures and written description are provided to teach any person skilled in the art how to make and use the inventions for which patent protection is sought. Those skilled in the art will appreciate that not all features of a commercial embodiment of the inventions are described or shown for the sake of clarity and understanding. Persons of skill in this art will also appreciate that the development of an actual commercial embodiment incorporating aspects of the present disclosure will require numerous implementation-specific decisions to achieve the developer’s ultimate goal for the commercial embodiment. Such implementation-specific decisions may include, and likely are not limited to, compliance with system-related, business-related, government-related, and other constraints, which may vary by specific implementation, location, or with time. While a developer’s efforts might be complex and time-consuming in an absolute sense, such efforts would be, nevertheless, a routine undertaking for those of ordinary skill in this art having benefit of this disclosure. It must be understood that the inventions disclosed and taught herein are susceptible to numerous and various modifications and alternative forms. The use of a singular term, such as, but not limited to, “a,” is not intended as limiting of the number of items. Further, the various methods and embodiments of the system can be included in combination with each other to produce variations of the disclosed methods and embodiments. Discussion of singular elements can include plural elements and vice-versa. References to at least one item may include one or more items. Also, various aspects of the embodiments could be used in conjunction with each other to accomplish the understood goals of the disclosure. Unless the context requires otherwise, the term “comprise” or variations such as “comprises” or “comprising,” should be understood to imply the inclusion of at least the stated element or step or group of elements or steps or equivalents thereof, and not the exclusion of a greater numerical quantity or any other element or step or group of elements or steps or equivalents thereof. The device or system may be used in a number of directions and orientations. The terms “top”, “up’, “upward’, “bottom”, “down”, “downwardly”, and like directional terms are used to indicate the direction relative to the figures and their illustrated orientation and are not absolute relative to a fixed datum such as the earth in commercial use. The term “inner,” “inward,” “internal” or like terms refers to a direction facing toward a center portion of an assembly or component, such as longitudinal centerline of the assembly or component, and the term “outer,” “outward,” “external” or like terms refers to a direction facing away from the center portion of an assembly or component. The term “coupled”, “coupling”, “coupler”, and like terms are used broadly herein and may include any method or device for securing, binding, bonding, fastening, attaching, joining, inserting therein, forming thereon or therein, communicating, or otherwise associating, for example, mechanically, magnetically, electrically, chemically, operably, directly or indirectly with intermediate elements, one or more pieces or members together and may further include without limitation integrally forming one functional member with another in a unitary fashion. The coupling may occur in any direction, including rotationally. The order of steps can occur in a variety of sequences unless otherwise specifically limited. The various steps described herein can be combined with other steps, interlineated with the stated steps, and/or split into multiple steps. Similarly, elements have been described functionally and can be embodied as separate components or can be combined into components having multiple functions. Some elements are nominated by a device name for simplicity and would be understood to include a system of related components that are known to those with ordinary skill in the art and may not be specifically described. Some elements are nominated by a device name for simplicity and would be understood to include a system of related components that are known to those with ordinary skill in the art and may not be specifically described. Various examples are provided in the description and figures that perform various functions and are non-limiting in shape, size, description, but serve as illustrative structures that can be varied as would be known to one with ordinary skill in the art, given the teachings contained herein. As such, the use of the term “exemplary” is the adjective form of the noun “example” and likewise refers to an illustrative structure, and not necessarily a preferred embodiment. Element numbers with suffix letters, such as “A”, “B”, and so forth, or numbers with prime, double prime, and so forth, such as 1, 1′, 1″, and so forth, are to designate different elements within a group of like elements having a similar structure or function, and corresponding element numbers without the letters are to generally refer to one or more of the like elements. Any element numbers in the claims that correspond to elements disclosed in the application are illustrative and not exclusive, as several embodiments are disclosed that use various element numbers for like elements. Unless otherwise defined, all technical and scientific terms used herein have the same meaning as commonly understood by one of ordinary skill in the art to which this invention belongs. Although methods, materials, and/or systems similar or equivalent to those described herein can be used in the practice of the present disclosure, suitable methods, materials, and/or materials are described below. In addition, the methods, materials, and/or systems are illustrative and not intended to be limiting, unless stated otherwise. Publications, patents, and other references mentioned herein are incorporated by reference in their entirety, provided that in case of conflict, the present specification, including definitions, will control.

Generally, the invention provides a method of increasing the performance of microalgae and organisms with conserved metabolic pathways compared to untreated organisms when maintained under normal conditions, and in particular the yield of lipids, biomass, and metabolites. The method comprises the use of electromagnetic ionizing radiation to induce rapid and reproducible metabolic activation via the hormetic effects of irradiation that is applied at specific absorbed doses and rates to organisms. In at least one embodiment, the irradiation is applied in the exponential or stationary phase of microalgae growth. The hormetic effect of electromagnetic ionizing radiation involves the up-regulation of expression of lipid metabolism genes encoding enzymes that are involved in the biosynthesis of lipids and accumulation of energy reserves in the form of lipids and/or the accumulation of other different metabolites and/or an increase in the biomass. The method can be implemented using existing biotechnology cultivation platforms, including but not limiting to raceway ponds, photo-bioreactors, airlift bioreactors, bubble column bioreactors, fermentation bioreactors, biofilm reactors, and suspended or biofilm cultivation for microalgae and organisms grown on other cultivation/growth substrate, standard cultivation conditions, including but not limiting to photoautotrophic, heterotrophic and mixotrophic cultivation, standard culture types, including but not limiting to batch, semi-continuous, and continuous, and other platforms. Applicable organisms include, without limitation, classes of microalgae strains including freshwater and marine algae, such as without limitation, Bacillariophyta (diatoms), Chlorophyta (green algae), Cyanobacteria (blue-green algae), Haptophyta (mostly marine algae including coccoliths), Phaeophyta (brown algae), Pyrrophycophyta (dinoflagellates), Rhodophyta (red algae), and organisms with conserved metabolic pathways, such as without limitation, Bigyra (including Thraustochytrids), Streptophyta (plants including mosses), Ascomycota (fungi including yeasts), Basidiomycota (fungi including mushrooms), bacteria, and archaea. The present invention can interface with typical cells with at least one of X-ray and gamma radiation that produces hormesis in cells, and metabolic activation of the cells of such organisms to produce a hormetic effect in the culture under given conditions compared to an untreated culture of the same strain of such cells when maintained under the same conditions. Generally, an irradiation system is positioned in operational proximity to an organism to produce the hormetic effect described herein.

For purposes herein, the following terms and their derivatives apply.

“Biomass yield” refers to a dry mass of the cell population harvested per volume of microalgae culture. Harvesting biomass means separating cells from an aqueous medium. Increased biomass yield generally is a result of increased productivity of biomass, where productivity is intended to mean the rate at which biomass is produced.

“Drying” means substantively removing the water content from harvested biomass.

“Electromagnetic ionizing radiation” refers to electromagnetic radiation of sufficient energy to induce lysis of water molecules, such as X-rays and gamma rays. Irradiation with electromagnetic ionizing radiation includes a uniform exposure of microalgae culture or another organism to a specific dose of radiation at a specific rate. The dose can be delivered continuously or in segments. One such source of radiation can be an X-ray tube with selected voltage, current, and filter(s) to deliver one or more specific doses and rates.

“Exponential phase” refers to an early period in microalgae culture growth showing exponential increase of cell density or biomass, and for the purpose of this invention includes lag phase, early exponential phase, and late exponential phase, which is known by a person with ordinary skill in the art.

“Growth rate” refers to the rate change in cell density or in biomass per day. Cell density shows a correlation coefficient close to 1 with optical density at 750 nm (OD₇₅₀) that is established by a photometric method that measures OD₇₅₀, which is known by a person with ordinary skill in the art.

“Hormesis” refers to a biological phenomenon whereby beneficial effects, such as improved growth, stress tolerance, or some other priming-related metabolic activation (e.g. accumulation of energy reserves or accumulation of a certain metabolite), result from the exposure to low doses of an agent that is otherwise toxic or lethal when given at higher doses. These beneficial effects of hormesis are due to “metabolic activation” that may involve mRNA transcription, post-transcriptional modifications, and metabolic pathways regulation, but are not due to genetic mutations.

“Lipid metabolism genes” refers to genes that encode enzymes that are involved in lipid metabolism, including fatty acid and triacylglycerol synthesis.

“Lipid yield” refers to the total mass of lipids in biomass that is harvested per volume of culture. Common lipids include triglycerides, diglycerides, free fatty acids, and phospholipids. Lipids can be extracted by standard methods that include mechanical crushing or sonication and extraction with organic solvents and heating or with supercritical carbon dioxide, which are known and available to persons skilled in the art. The increased lipid yield is a result of increased productivity of lipids. Lipid yield is different from lipid content, which refers to the percentage of lipids within the biomass and does not take into account harvested biomass yield that may be significantly reduced with prior methods of irradiation that can results in a net loss of lipid yield from the reduced biomass.

“Metabolite yield” refers to the total mass of metabolites in biomass that is harvested per volume of culture. Common metabolites include but are not limited to pigments, antioxidants, nutrients, drug precursors, omega-3 fatty acids, proteins, carbohydrates, and other products of primary and secondary metabolism. Metabolites can be extracted by standard methods that include but are not limited to mechanical crushing and extraction with selected solvents and heating, which are known to a person with ordinary skill in the art.

“Microalgae” refers to unicellular eukaryotic and prokaryotic microorganisms that are photosynthetic or derived from a phylogenetically photosynthetic heritage and that exists individually or in chains, groups or colonies, including but not limited to green algae, dinoflagellates, diatoms, brown algae, marine algae (including coccoliths), cyanobacteria, and red algae.

“Microalgae culture” refers to microalgae grown in aqueous medium or on solid medium under normal conditions for commercial, research, or other applications.

“Normal conditions” refers to conditions that are generally known by those skilled in the art including but not limited to recommended conditions for photoautotrophic, heterotrophic or mixotrophic cultivation of a particular microalgae strain or cultivation of organisms with conserved metabolic pathways for a specific cultivation platform and culture type. Normal conditions include commonly used media or growth substrates with no intentional limitation of nutrients, and recommended temperature range, nutrient concentration, light intensity, and regime that are known by the persons skilled in the art.

“Organisms with conserved metabolic pathways” refers to the presence of functionally equivalent genes that regulate the metabolism in a similar manner as in microalgae, including but not limited to plants, fungi, yeast, thraustochytrids, bacteria, and archaea.

“Stationary phase” of growth refers to a late period in microalgae culture growth that generally shows a growth rate that is close to zero or shows day-to-day variations around a zero value. The stationary phase follows the exponential phase, and for the purpose of this invention includes pre-stationary phase, early stationary phase, late stationary phase, and death phase (or lysis phase), which are known by a person with ordinary skill in the art.

“Up-regulation of transcription” refers to significantly increased number of copies of messenger RNA in response to a certain stimulus.

In at least one aspect of the present invention, there is provided a method of inducing metabolic activation in organisms for increased lipid yield without a substantial change (that is, a statistically insignificant change) in biomass yield through irradiation of the organisms in a hormesis manner compared to untreated organisms when maintained under the same normal conditions. The hormesis irradiation results in the up-regulation of expression of lipid metabolism genes, including genes of enzymes that synthesize lipids. Organisms include microalgae and organisms with conserved metabolic pathways relevant to lipid yield. The lipid yield includes the yield of omega-3 fatty acids. The method includes an irradiation exposure at certain growth periods of the organism, such as exposure of microalgae in the stationary phase of growth.

In at least another aspect of the present invention, there is provided a method of inducing metabolic activation in organisms, such as and not limited to microalgae and organisms with conserved metabolic pathways, that results in increased growth rate and biomass yield compared to the untreated culture when maintained under the same normal conditions. The method includes hormesis irradiation of the organism at certain growth periods, such as a microalgae culture in the exponential phase of growth.

The invention also features lipid and biomass yield that are increased by any of the methods described herein in microalgae cultures that are grown and/or maintained under abnormal conditions.

The invention also features the up-regulation of expression of lipid metabolism genes encoding enzymes that synthesize lipids and increase lipid yield in organisms with conserved metabolic pathways by any of the methods described herein.

The invention also features the regulation of genes that are involved in other metabolic pathways for the ability to produce other metabolites and products by any of the methods described herein.

For exemplary purposes, the discussion herein and embodiments focus on microalgae with the understanding that the methods and principles discussed herein can apply to the other organisms with conserved metabolic pathways.

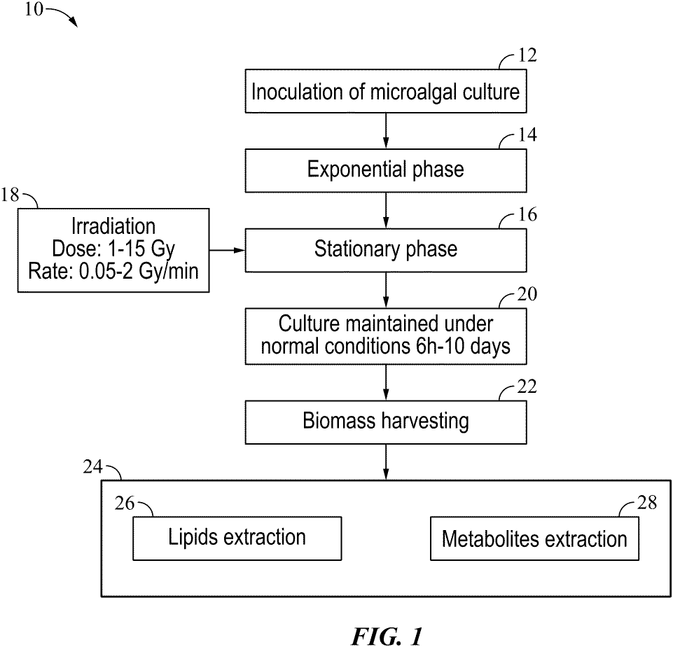

FIG. 1 is a flowchart for an example of at least one embodiment using a method of the invention in a stationary phase of growth of microalgae and other organisms with conserved metabolic pathways relevant to lipid yield.

In general, for at least one embodiment of the invention, a microalgae culture can be grown after initial inoculation into a medium under normal conditions to reach a stationary phase of growth. The culture can be briefly exposed while in the stationary phase to electromagnetic ionizing radiation, such as X-ray radiation. The culture can be maintained under normal conditions until biomass harvesting, where the microalgae cells are collected. At biomass processing, lipids can be extracted from the biomass. Using the teachings herein of the invention, irradiated microalgae cultures exhibit a higher lipid yield compared to an untreated culture of the same microalgae strain grown under the same conditions.

More specifically, the method 10 can include an inoculation step 12 where a microalgae culture can be formed by inoculation of cells into a medium that is recommended for the selected microalgae strain. The culture can be maintained under the normal conditions needed to undergo a set of growth phases. The culture can go into an exponential phase 14, which is followed by a stationary phase 16. Biomass is the highest in the stationary phase and does not change significantly during this phase. In an irradiation step 18, the microalgae culture can be irradiated during the stationary phase with an applied dose of electromagnetic ionizing radiation. The irradiation can be continuously applied or in segments. As an example and without limitation, the microalgae culture can be exposed to X-ray radiation in a total absorbed dose of 1 to 15 Gy, with rates of irradiation between 0.05 and 2 Gy of absorbed dose per minute. After irradiation, the microalgae can be maintained in maintenance step 20 for an optimal period of time from the irradiation, such as six hours to ten days. After the maintenance, the microalgae can undergo biomass harvesting step 22, including collecting the microalgae cells. After harvesting, biomass processing step 24 can include mechanically disruption of the microalgae cells. Lipids extraction step 26 and/or metabolites extraction step 28 can occur, such as by using a mixture of organic solvents and increased temperatures or using supercritical carbon dioxide extraction protocols that are specific for the selected microalgae culture and can be optimized by one with ordinary skill in the art.

Thus, in at least one embodiment, the presented invention provides a method of increasing lipid yield and/or metabolites yield in a culture compared to an untreated culture of the same strain when maintained under the same conditions.

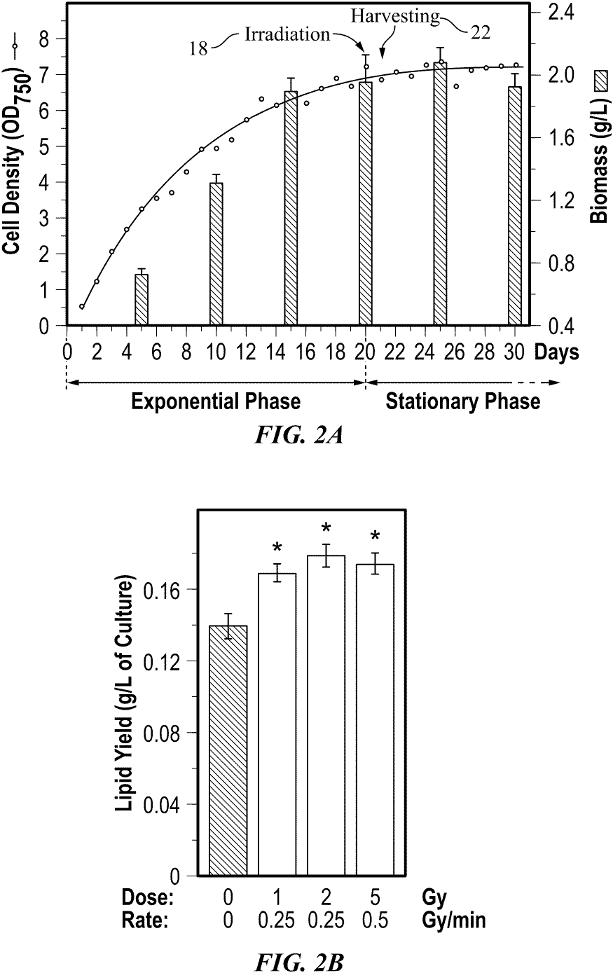

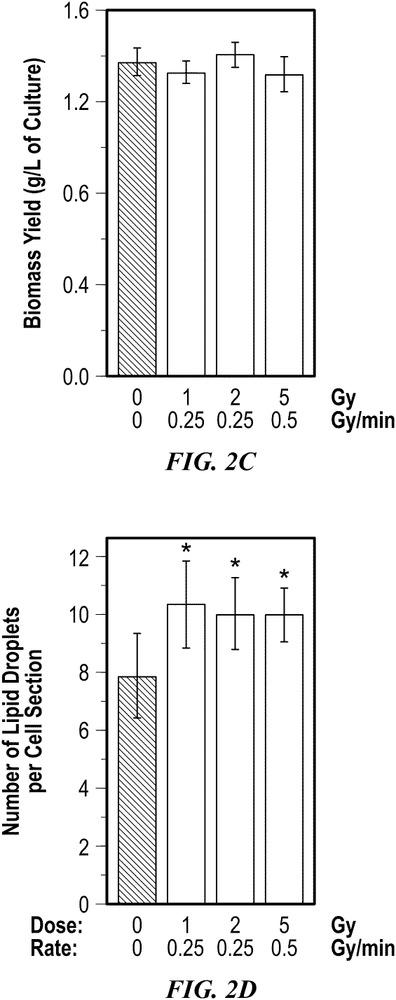

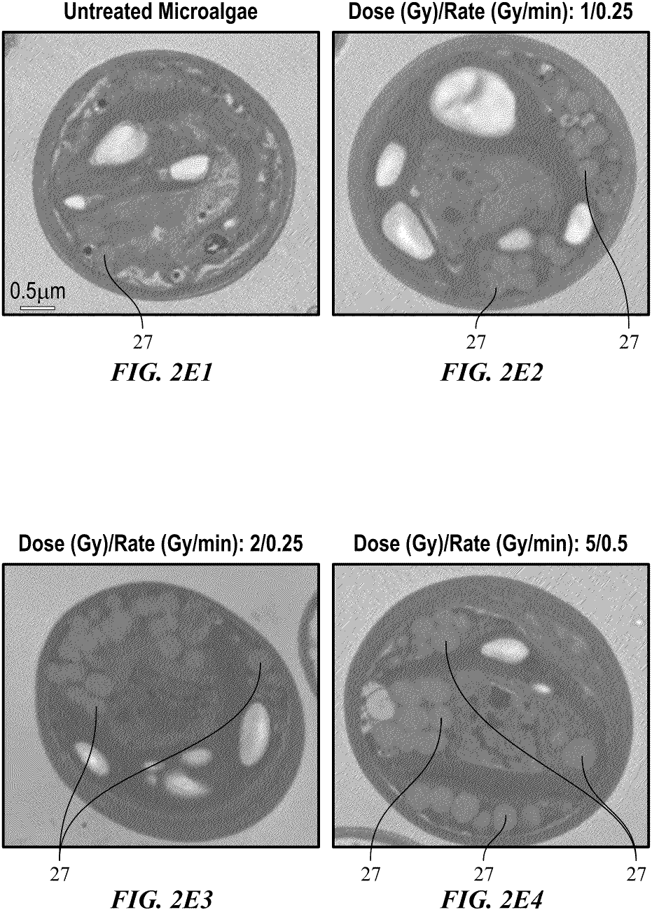

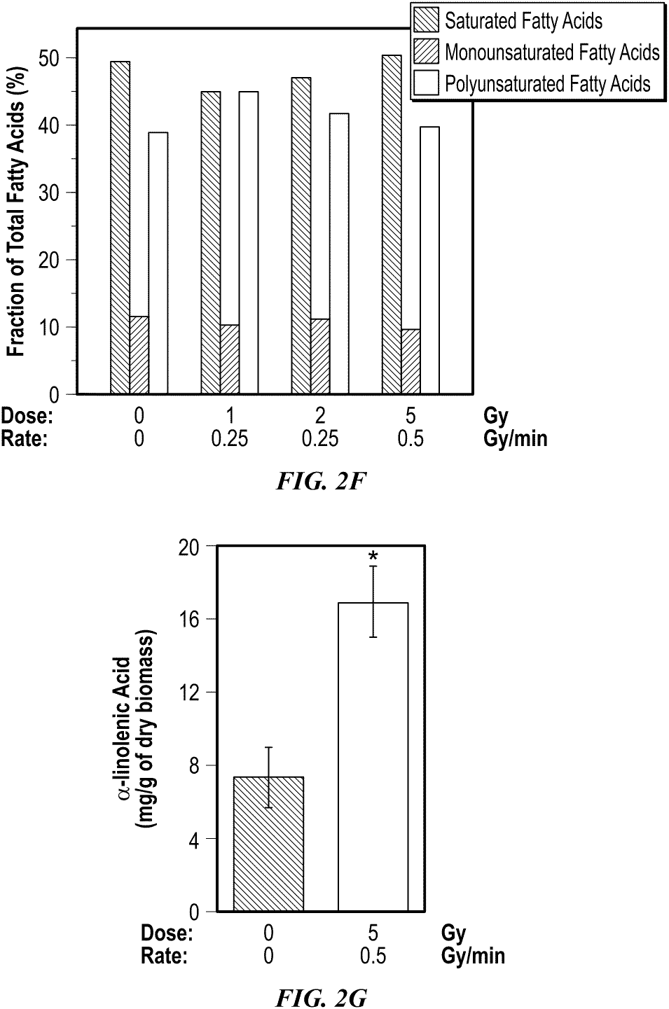

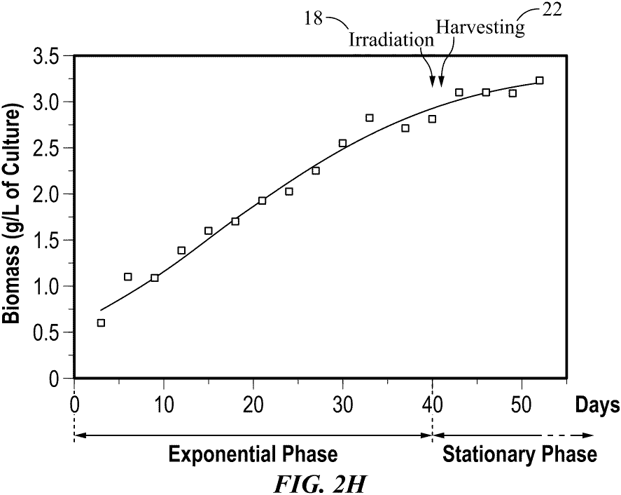

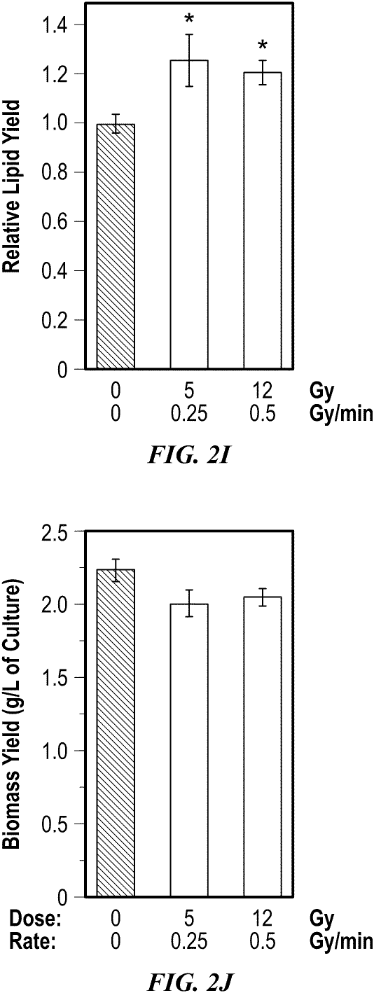

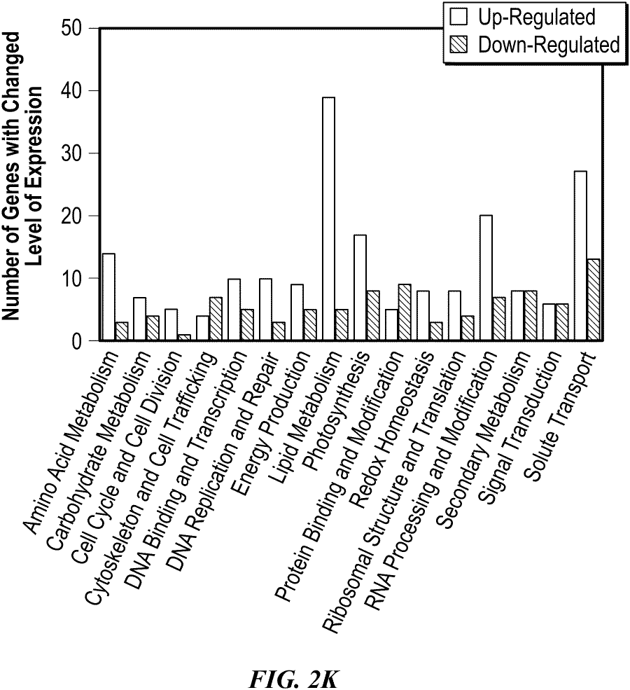

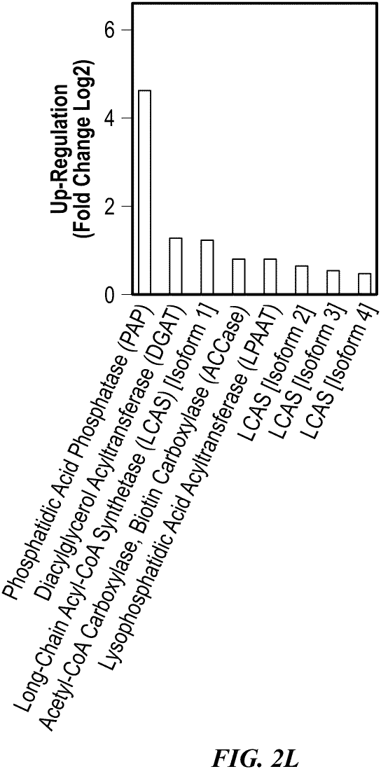

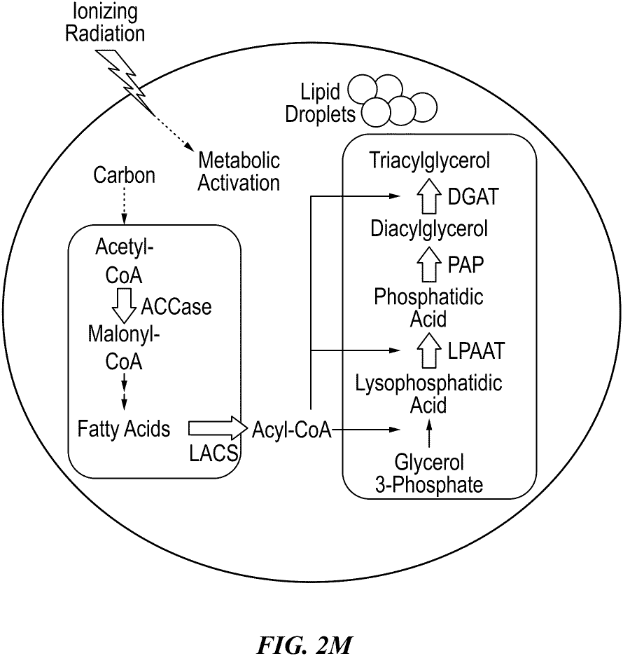

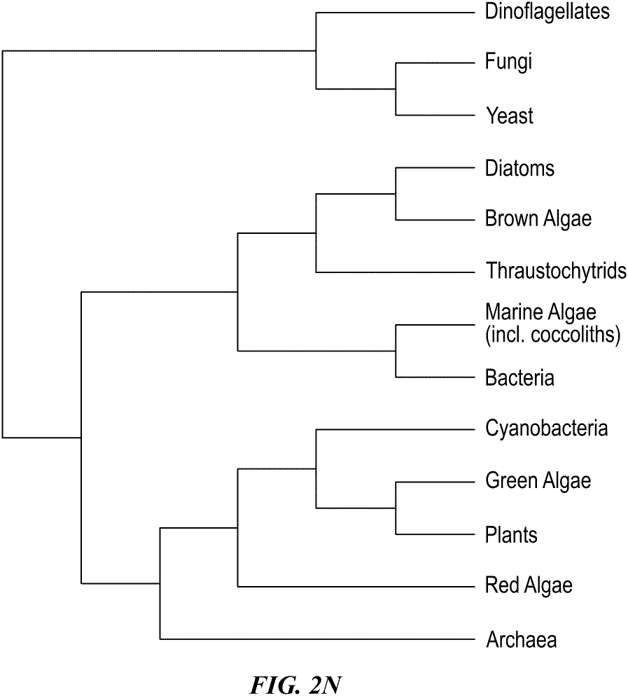

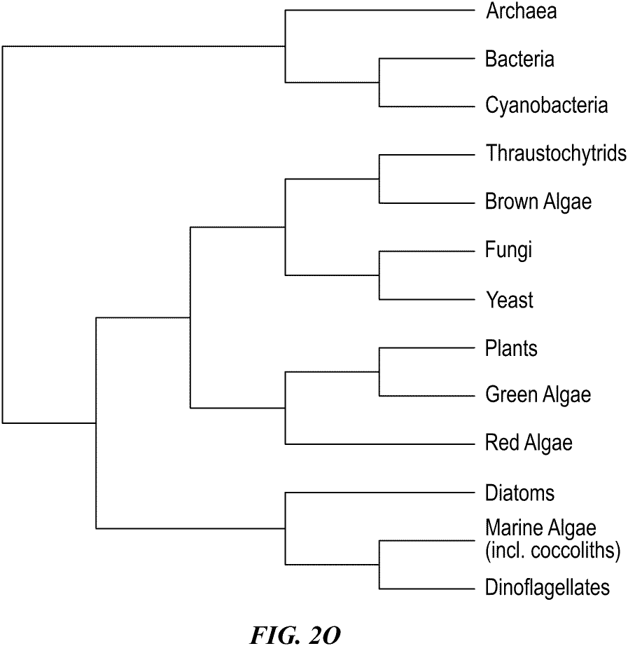

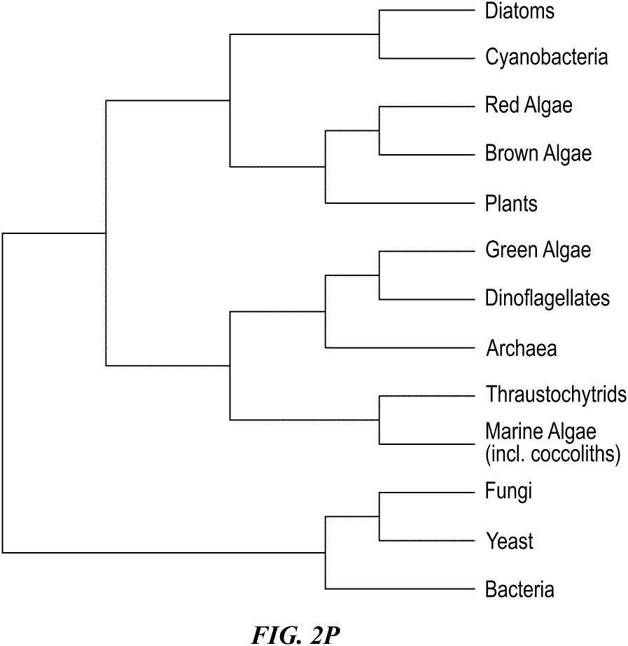

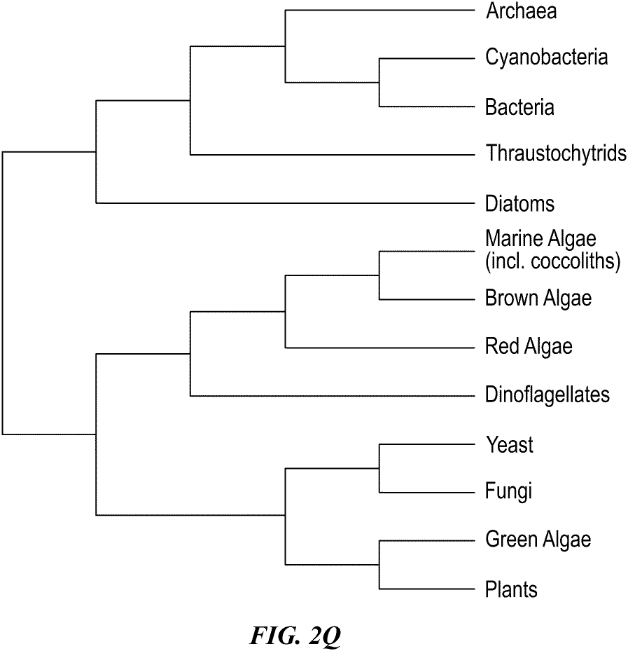

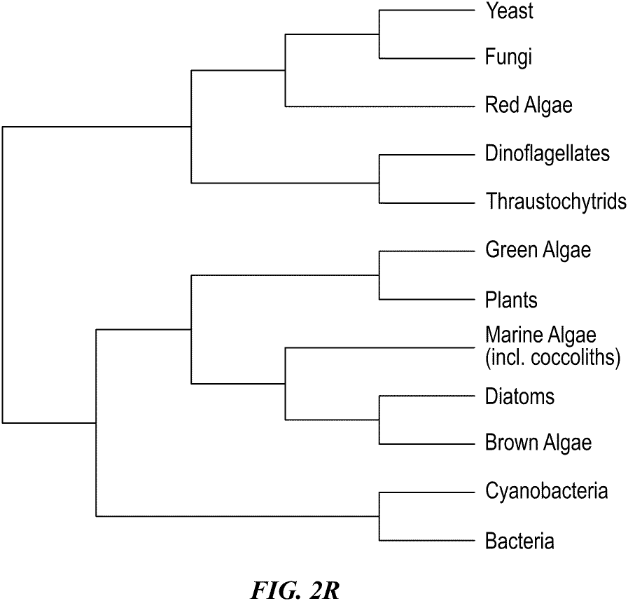

FIGS. 2A-2L are exemplary graphs, photos, and schematic diagrams of actual test data based on applying the electromagnetic ionizing radiation of the exemplary method of the invention in the stationary phase of microalgae growth under otherwise normal conditions.

### Experiment 1 for Stationary Phase

FIG. 2A is a graph of an example of a growth curve of an organism exposed to the hormesis X-ray radiation of the invention with the process of FIG. 1. FIG. 2B is a graph of an example of a statistically significant positive impact on lipid yield of the organism of FIG. 2A for given irradiation dosing at various dosing rates. FIG. 2C is a graph of an example of no significant negative impact on biomass yield of the organism of FIG. 2A for given irradiation dosing at various dosing rates.

A culture of microalga Chlorella sorokiniana (CCAP 211/8 K; alternative designation UTEX 1230), which is an example of a green algal strain, was grown under normal conditions in 3N-BBM+V medium (recipe available at https://www.ccap.ac.uk/media/documents/3N_BBM_V.pdf), at 22° C. on an orbital shaker (120 rpm), and a continuous photon flux of 120 µmol m⁻² s⁻¹. The microalgae culture was inoculated into the fresh medium with 0.5 × 10⁶ cells/mL, passed through the exponential phase with growth, and reached the stationary phase at day 20 (FIG. 2A). The microalgae culture was irradiated in an irradiation step 18 with X-rays at day 20, using a CellRad system (FaxitronBioptics LLC; tube power: 750 W; filtration: 1.6 mm Be and 0.5 mm AI; energy 120 kV; dose and rate were adjusted by changing the current). Absorbed doses were 1, 2, or 5 Gy. Rates of absorption were 0.25 or 0.5 Gy/min. Irradiated cultures were further maintained under the same normal conditions for 24 h. The biomass was harvested in a biomass harvesting step 22 on day 21 by centrifugation at 5000 g for 5 min and drying of the pellet for 24 h at 50° C. (The increased lipids are preserved for a significant period of time after the irradiation.) The biomass was mechanically crushed and lipids were extracted using chloroform and methanol 2/1 (v/v) mixture and soxhlet extractor working at 80° C. for 4 h. Extracted lipids were dried at 50° C. for 12 h. Irradiated samples showed a significant increase in lipid yield of 22% to 29% in just 24 h compared to an untreated culture that was grown under the same normal conditions (FIG. 2B). The irradiation did not induce a reduction of biomass yield (FIG. 2C).

FIG. 2D is a graph of an example of a statistically significant positive impact on lipid droplets of cells of the organism of FIG. 2A for given irradiation dosing at various dosing rates. FIG. 2E1 is micrograph of an untreated cell of the organism of FIG. 2A with limited number of lipid droplets. FIG. 2E2 is a micrograph of a treated cell of the organism of FIG. 2A with a given irradiation dose and rate showing increased lipid droplets over the untreated cell of the organism of FIG. 2E1. FIG. 2E3 is a micrograph of a treated cell of the organism of FIG. 2A with a given irradiation dose and rate showing increased lipid droplets over the untreated cell of the organism of FIG. 2E1. FIG. 2E4 is a micrograph of a treated cell of the organism of FIG. 2A with a given irradiation dose and rate showing increased lipid droplets over the untreated cell of the organism of FIG. 2E1.

Cells were collected from the same samples just before biomass harvesting and analyzed using transmission electron microscopy. Irradiated cells contained a larger number of lipid droplets 27 than untreated cells grown under the same normal conditions (FIG. 2D). The accumulation of lipid droplets 27 as a response to irradiation dosing shown in FIG. 2C was further confirmed by transmission electron microscopy by comparing the untreated cell (FIG. 2E1) with the treated cell with a dose of 1 Gy at a dose rate of 0.25 Gy per minute (FIG. 2E2); the treated cell with 2 Gy at 0.25/min. (FIG. 2E3); and the treated cell with 5 Gy at 0.5 Gy/min. (FIG. 2E4).

FIG. 2F is a graph of an example of no substantive impact on a fatty acids profile of cells of the organism of FIG. 2A for given irradiation dosing at various dosing rates. FIG. 2G is a graph of an example of a statistically significant positive impact on metabolite alpha-linoleic acid (omega-3 fatty acid) content in biomass of the organism of FIG. 2A for an exemplary irradiation dosing at a given dosing rate.

The fatty acid profile of the extracted lipids was determined by gas chromatography. The fatty acids in the extracted lipids were transesterified using a methanolic sulfuric acid protocol, collected with hexane and dissolved in dichloromethane for analysis. The irradiation did not induce changes in fatty acids profile (saturated fatty acids, monounsaturated fatty acids, and polyunsaturated fatty acids), and the level of saturation (FIG. 2F), both of which represent parameters that are relevant for biodiesel production and other uses. Cells irradiated with 5 Gy at 0.5 Gy/min contained increased amount of alpha-linoleic acid than untreated cells grown under the same normal conditions (FIG. 2G). Alpha-linoleic acid is an omega-3 fatty acid that is essential for human nutrition and a precursor for other omega-3 fatty acids.

### Experiment 2 for Stationary Phase

FIG. 2H is a graph of an example of a growth curve of another organism exposed to the X-ray radiation of the invention with the process of FIG. 1. FIG. 2I is a graph of an example of a statistically significant positive impact on lipid yield of the organism of the FIG. 2H for given irradiation dosing at various dosing rates. FIG. 2J is a graph of an example of no significant negative impact on biomass yield of the organism of the FIG. 2H for given irradiation dosing at various dosing rates.

In a second experiment, the culture of microalga Chlamydomonas reinhardtii (strain CCAP1 1/32C) was grown under normal conditions in TAP medium (recipe available in Harris, E.H. (1989) The Chlamydomonas Sourcebook. Academic Press, San Diego), at 22° C. on an orbital shaker (120 rpm), with a continuous photon flux of 120 µmol m⁻² s⁻¹. The microalgae culture was inoculated with 2 × 10⁶ cells/mL, passed through the exponential phase with growth, and reached the stationary phase at day 40 (FIG. 2H). Microalgae cultures were irradiated in an irradiation step 18 with X-rays at day 40 using CellRad system. Absorbed doses were 5 or 12 Gy with rates of absorption of 0.25 or 0.5 Gy/min, respectively. Irradiated samples were further grown for 24 h at the same normal conditions. The biomass was harvested (step 22). The relative lipid yield was determined in microalgae culture using Nile Red fluorescent dye assay. The fluorescence intensity of this dye with excitation at 530 nm and emission at 570 nm in microalgae culture is proportional to the amount of lipids per volume. The fluorescence of samples of irradiated cultures were normalized to the fluorescence of non-irradiated cultures to determine relative lipid yield. The irradiation of microalgae culture with X-rays induced significant increase in lipid yield of about 21% to 26% compared to untreated culture that was grown under the same normal conditions (FIG. 2I). The irradiation had no significant negative effects on biomass yield (FIG. 2J), which was harvested by centrifugation at 5000 g for 5 min and drying of the pellet for 24 h at 50° C.

### Experiment 3 for Gene Expression

FIG. 2K is a graph of an example of positive impact of irradiation of the invention on up-regulation of expression of different classes of genes, including lipid metabolism genes in the organism of FIG. 2A. FIG. 2L is a graph of an example of up-regulation of expression of lipid metabolism genes for the production of fatty acids and triacylglycerols in lipid droplets in the organism of FIG. 2A. FIG. 2M is a diagram explaining an exemplary of the process of metabolic activation and up-regulation of expression of lipid metabolism genes for the production of fatty acids and triacylglycerols in lipid droplets for the irradiation on a cell of an organism of FIG. 2A. FIG. 2N is a diagram of an example of a phylogenetic tree of a lipid metabolism gene Acetyl-CoA carboxylase, biotin carboxylase (ACCase) for the synthesis of fatty acids in lipid droplets, including from the green algae organism of FIG. 2A and FIG. 2H, showing high evolutionary conservation across classes of microalgae and organisms with conserved metabolic pathways, including plants, yeast, fungi, bacteria and archaea. FIG. 2O is a diagram of an example of a phylogenetic tree of a lipid metabolism gene Long-chain acyl-CoA synthetase (LACS) for the synthesis of fatty acids in lipid droplets, including from the green algae organism of FIG. 2A, showing high evolutionary conservation across classes of microalgae and organisms with conserved metabolic pathways, including plants, yeast, fungi, bacteria and archaea. FIG. 2P is a diagram of an example of a phylogenetic tree of a lipid metabolism gene Lysophosphatidic acid acyltransferase (LPAAT) for the synthesis of triacylglycerols in lipid droplets, including from the green algae organism of FIG. 2A, showing high evolutionary conservation across classes of microalgae and organisms with conserved metabolic pathways, including plants, yeast, fungi, bacteria and archaea. FIG. 2Q is a diagram of an example of a phylogenetic tree of a lipid metabolism gene Phosphatidic acid phosphatase (PAP) for the synthesis of triacylglycerols in lipid droplets, including from the green algae organism of FIG. 2A, showing high evolutionary conservation across classes of microalgae and organisms with conserved metabolic pathways, including plants, yeast, fungi, bacteria and archaea. FIG. 2R is a diagram of an example of a phylogenetic tree of a lipid metabolism gene Diacylglycerol acyltransferase (DGAT) for the synthesis of triacylglycerols in lipid droplets, including from the green algae organism of FIG. 2A, showing high evolutionary conservation across classes of microalgae and organisms with conserved metabolic pathways, including plants, yeast, fungi, bacteria and archaea.

The gene expression profile and the identification of transcriptionally up- and down-regulated metabolism genes as a response to 5 Gy irradiation at 0.5 Gy/min in C. sorokiniana, for example, was determined by sequencing of extracted messenger RNA (mRNA). RNA extraction was performed by the incubation with TRIzol Reagent and chloroform, centrifugation for 15 min at 12,000 g and 4° C. and collection of the upper layer, and RNA precipitation with isopropanol. The RNA pellet was washed two times with and centrifugation at 12,000 g/5 min at 4° C. and resuspended in sterile deionised H₂O. RNA libraries were generated using the TruSeq® Stranded mRNA assay according to the protocol of manufacturer Illumina, Inc. Irradiated cells showed the up-regulated expression of over 30 lipid metabolism genes, including genes encoding the key enzymes for the synthesis of triacylglycerols that accumulate in lipid droplets, as well as a large number of up-regulated genes related to other important metabolism and cellular functions (FIGS. 2K-2M). The evolutionary conservation of the lipid metabolism genes showing up-regulated expression in response to irradiation from this green algae strain, was determined by performing gene sequence searches for related genes from other microalgae organisms, including red algae, brown algae, marine algae (including coccoliths), diatoms, dinoflagellates, and cyanobacteria, and other organisms with conserved metabolic pathways, including thraustochytrids, plants, yeast, fungi, bacteria and archaea, using the Basic Local Alignment Search Tool. Amino acid sequence alignment and phylogenetic analysis by maximum likelihood was performed to generate a set of phylogenetic trees for each gene. The up-regulated genes encoding the key enzymes for the synthesis of fatty acids and triacylglycerols and their regulation (FIG. 2L) are shown to be highly evolutionarily conserved in microalgae and organisms with conserved metabolic pathways, including but not limited to macroalgae and plants (FIGS. 2N-2R).

Comparison of the acetyl-CoA carboxylase (ACCase) gene from the exemplar green alga strain Chlorella sorokiniana with representative genes from the other organisms tested, found 46% average sequence similarity, with many organisms showing high sequence similarity ranging between 43 - 72% (FIG. 2N). For example, ACCase genes from the red alga Porphyra umbilicalis and from the cyanobacterium Nostoc punctiforme showed 58% and 68% sequence similarity, respectively with C. sorokinianai ACCase. Comparison of the long-chain acyl-CoA synthetase (LACS) gene from C. sorokinianai (green algae) with LACS genes from the other organisms showed high sequence similarity values ranging between 36 - 64% (FIG. 2O). For example, LACS genes from the Thraustochytrid Schizochytrium aggregatum and the plant Arabidopsis thaliana showed 52% and 64% sequence similarity, respectively with C. sorokinianaii LACS. Comparison of the lysophosphatidic acid acyltransferase (LPAAT) gene from C. sorokiniana (green algae) with LPAAT genes from the other organisms showed high sequence similarity values ranging between 35 - 43% (FIG. 2P). For example, LPAAT genes from the yeast Saccharomyces cerevisiae and the dinoflagellate Symbiodinium microadriaticum showed 40% and 43% sequence similarity, respectively with C. sorokinianaii LPAAT. Comparison of the phosphatidic acid phosphatase (PAP) gene from C. sorokinianaii (green algae) with PAP genes from the other organisms showed high sequence similarity values ranging between 33 - 46% (FIG. 2Q). For example, PAP genes from the brown alga Ectocarpus siliculosus and the plant Beta vulgaris showed 40% and 46% sequence similarity, respectively with C. sorokinianaii PAP. Comparison of the diacylglycerol acyltransferase (DGAT) gene from C. sorokinianaii (green algae) with DGAT genes from the other organisms showed average sequence similarity of 42% and high sequence similarity values ranging between 35 - 54% (FIG. 2R). For example, DGAT genes from the diatom Phaeodactylum tricornutum and the fungus Rhizoctonia solani showed 43% and 45% sequence similarity, respectively with C. sorokinianaii DGAT. All of these high sequence similarity values (>40%) are demonstrating high evolutionary conservation across the different classes of organisms examined.

It can be further demonstrated that these lipid metabolism genes in other microalgae and in organisms with conserved metabolic pathways can also be up-regulated in response to stress conditions and are involved in the accumulation of triacylglycerols that accumulate in lipid droplets under stress. For example, in a diatom during salinity stress, genes including ACCase, LACS, and DGAT are up-regulated (Cheng, R.L., et al., 2014, Transcriptome and gene expression analysis of an oleaginous diatom under different salinity conditions. BioEnergy Research, vol. 7, pp. 195-205). For example, in a red algae genes including PAP and DGAT are up-regulated under nitrogen starvation (Imamura, S., et al., 2015, Target of rapamycin (TOR) plays a critical role in triacylglycerol accumulation in microalgae. Plant Molecular Biology, vol. 89, pp. 309-318). For example, in a dinoflagellate in response to nitrogen starvation, genes including DGAT are up-regulated (Shi, X., et al., 2021, Transcriptome responses of the dinoflagellate Karenia mikimotoi driven by nitrogen deficiency. Harmful Algae, vol. 103, article 101977). For example, in a filamentous fungus, genes required for TAG metabolism including LPAAT and DGAT are up-regulated in response to nitrogen starvation (Chen, Y., et al., 2018, Nitrogen-starvation triggers cellular accumulation of triacylglycerol in Metarhizium robertsii. Fungal Biology vol. 122, pp. 410-419). For example, in a Mycobacterium, DGAT genes required for triacylglycerol accumulation are induced by stresses including acid and hypoxia stress (Sirakova, T.D., et al., 2006, Identification of a diacylglycerol acyltransferase gene involved in accumulation of triacylglycerol in Mycobacterium tuberculosis under stress. Microbiology, vol. 152, pp. 2717-2725). The mechanisms of metabolic activation by ionizing radiation regulation (FIG. 2M) will involve the production of reactive oxygen species that can act as signaling molecules, and that can regulate metabolic pathways by transcription factor activation. Pathways of reactive oxygen species dependent signaling in response to stresses are documented in many organisms including plants and algae (for example, as described by Foyer, C.H. et al., 2018, Redox regulation of cell proliferation: bioinformatics and redox proteomics approaches to identify redox-sensitive cell cycle regulators. Free Radical Biology and Medicine, vol. 122, pp. 137-149; and Pokora, W., et al., 2022, Cross talk between hydrogen peroxide and nitric oxide in the unicellular green algae cell cycle: how does it work? Cells, vol. 11, article 2425). At least 10 transcription factor genes were significantly up-regulated in the irradiated microalgae (grouped within ‘DNA binding and transcription’, FIG. 2K) and coincident to the up-regulation of the other metabolism and cellular function genes. These Chlorella sorokiniana transcription factors belong to transcription factor gene families that are conserved across life. Collectively, this provides evidence that the mechanism of increase in metabolism genes in response to ionizing radiation and the regulation of metabolic activation is highly conserved across microalgae and organisms with conserved metabolic pathways.

### Experiment 4 for Exponential Phase

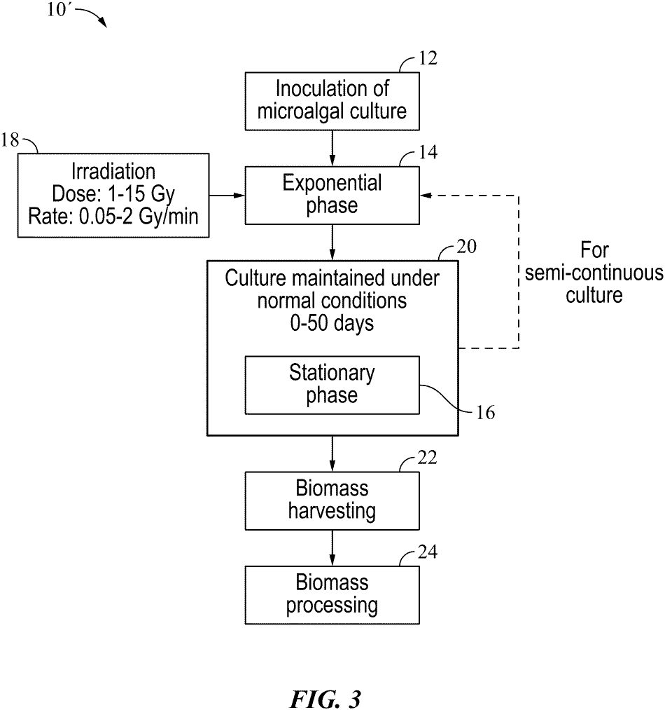

FIG. 3 is a flowchart for an example of at least one embodiment using a method of the invention in an exponential phase of growth of microalgae and other organisms with conserved metabolic pathways relevant to lipid yield. In general, for at least one embodiment of the invention, a microalgae culture can be grown after initial inoculation into a medium under normal conditions to reach an exponential phase of growth. The culture can be briefly exposed while in the exponential phase to electromagnetic ionizing radiation, such as X-ray radiation. The culture can be grown further under normal conditions until biomass harvesting of the complete culture is performed in batch culture type, or biomass harvesting of a part of the culture is performed, such as in semi-continuous culture type and the rest of the culture is left to grow to be irradiated again. The biomass can be processed according to the requirements of further use. Using the teachings herein of the invention, irradiated microalgae cultures exhibit a higher growth rates and biomass yield compared to an untreated culture of the same microalgae grown under the same conditions.

More specifically, the method 10′ can include an inoculation step 12, where a microalgae culture can be formed by inoculation of cells into a medium that is recommended for the selected microalgae strain. The culture can be maintained under the normal conditions needed to undergo a set of growth phases. The culture can go into an exponential phase 14, when an irradiation step 18 is applied generally in the early stages of the exponential phase. The microalgae culture can be irradiated with an applied dose of electromagnetic ionizing radiation. The irradiation can be continuously applied or in segments. As an example and without limitation, the microalgae culture can be exposed to X-ray radiation as a dose of about 1 to 15 Gy at rates between about 0.05 and 2 Gy/min. The microalgae culture can be maintained in maintenance step 20 under normal conditions of an additional 1 to 50 days, including transitioning into a stationary phase 16. After the maintenance period, biomass harvesting 22 is performed. After harvesting, biomass processing 24 is performed and can include any processing according to the requirements of further use.

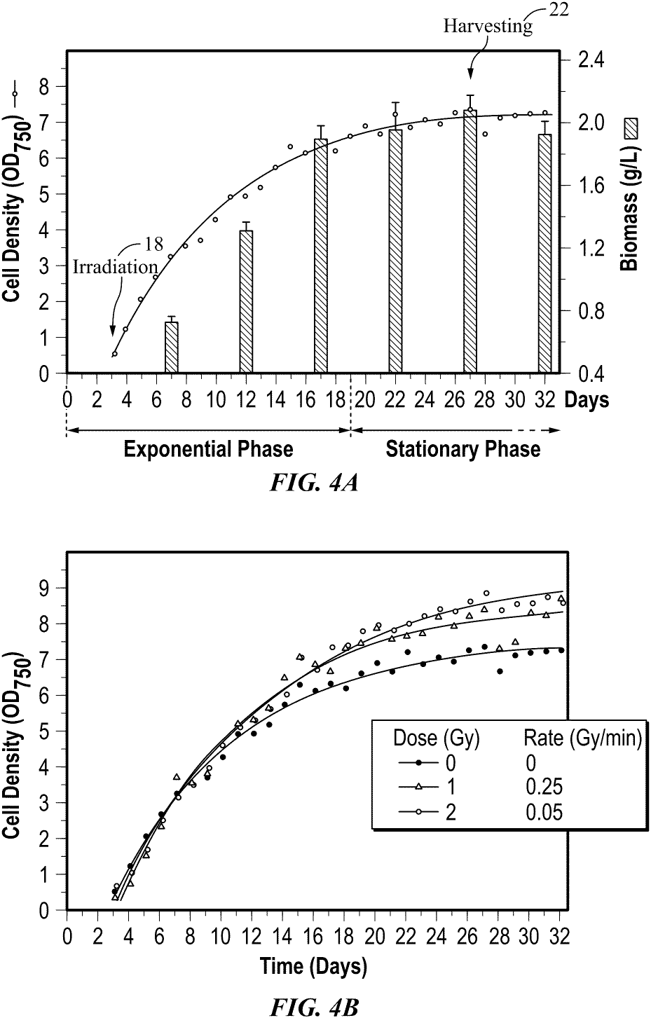

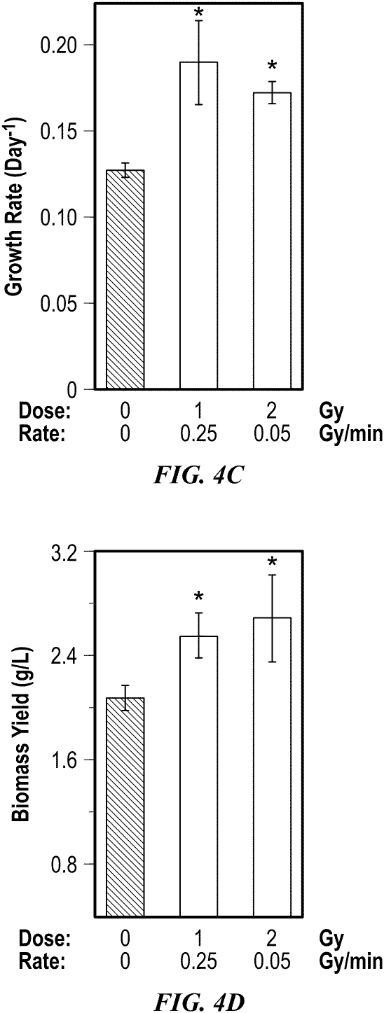

FIGS. 4A-4D are exemplary graphs of actual test data based on applying the electromagnetic ionizing radiation of the exemplary method of the invention in the exponential phase of microalgae growth under otherwise normal conditions. FIG. 4A is a graph of an example of a growth curve of the organism of FIGS. 2A-2G, but exposed to the hormesis X-ray radiation of the invention with the process of FIG. 3. FIG. 4B is a graph of an example of a positive impact on cell density of the organism of FIG. 4A for given irradiation dosing at various dosing rates. FIG. 4C is a graph of a statistically significant positive impact on growth rate of the organism of FIG. 4A for given irradiation dosing at various dosing rates. FIG. 4D is a graph of an example of a statistically significant positive impact on biomass yield of the organism of FIG. 4A for given irradiation dosing at various dosing rates.

The culture of microalga Chlorella sorokiniana (CCAP 211/8 K; alternative designation UTEX 1230) was grown under normal conditions in 3N-BBM+V medium, at 22° C. on an orbital shaker (120 rpm) and a continuous photon flux of 120 µmol m⁻² s⁻¹. The microalgae culture was inoculated with 0.5 × 10⁶ cells/mL. The microalgae culture was irradiated in an irradiation step 18 with X-rays at day 3 of the exponential phase, using a CellRad system. Absorbed doses were 1 or 2 Gy. Rates of absorption were 0.25 or 0.05 Gy/min. Irradiated samples were further grown for 24 days at the same normal conditions. Cell density was analyzed each day (FIG. 4A). Irradiation of microalgae culture in the exponential phase induced a positive impact on growth rate (FIGS. 4B-4C). The biomass was harvested in a biomass harvesting step 22 in the stationary phase, 25 days after irradiation, by centrifugation at 5000 g for 5 min and drying of the pellet for 24 h at 50° C. The biomass yield was increased by 25% in irradiated microalgae cultures when compared to untreated cultures of the same microalgae strain grown under the same conditions (FIG. 4D).

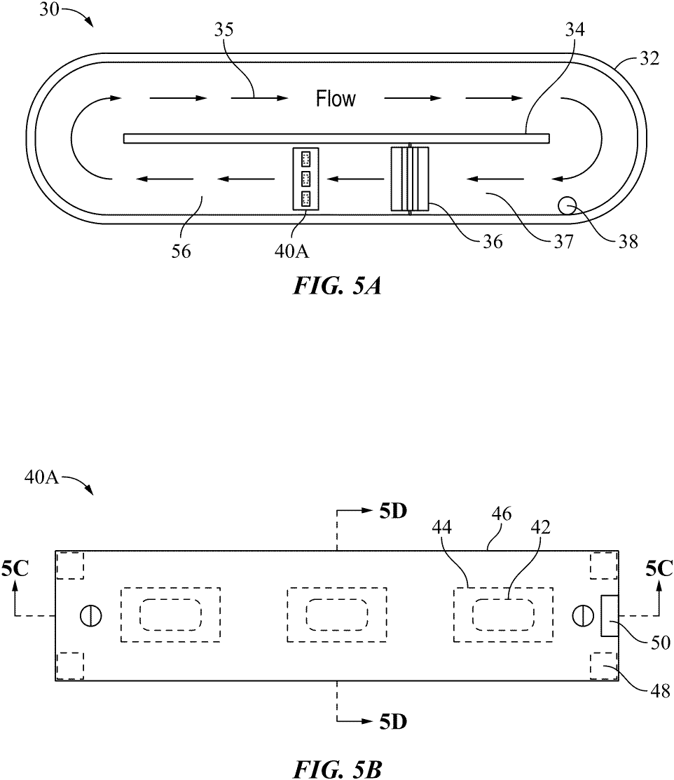

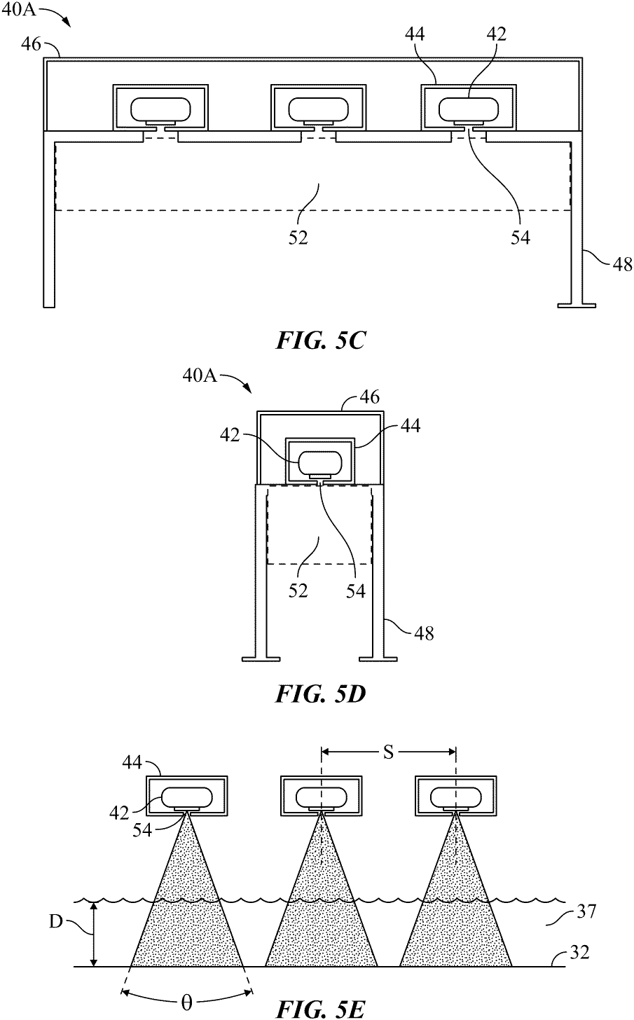

FIGS. 5A-10C illustrate various embodiments of systems using the inventive aspects described herein for microalgae and organisms with conserved metabolic pathways. Growth substrates for the microalgae and organisms with conserved metabolic pathways can include inorganic and organic materials and nutrients,.

FIGS. 5A-5E are illustrative schematic diagrams of at least one embodiment of an irradiation system of the invention configured to apply an exemplary method to an existing microalgae cultivation platform of a raceway pond under otherwise normal conditions. FIG. 5A is a top view schematic diagram of an example of an irradiation system that is mounted on a raceway pond. FIG. 5B is an enlarged top view schematic diagram of the irradiation system of FIG. 5A. FIG. 5C is an enlarged side sectional view schematic diagram through a longitudinal section of the irradiation system of FIG. 5A. FIG. 5D is an enlarged side sectional view schematic diagram through a lateral section of the irradiation system of FIG. 5A. FIG. 5E is an enlarged side sectional view schematic diagram of irradiation beam coverage through a longitudinal section of the irradiation system of FIG. 5A.

A standard raceway pond platform is known to one of ordinary skill in the art, and generally includes a container 32 having a growth substrate 56 of a volume of water split into channels (manufactured or formed in the ground) by a central baffle 34 to provide a flow circuit 35 around the container, a motive force 36, such as a paddlewheel, to move a microalgae culture 37 in the container around the flow circuit, and a sensor 38 to sense culture conditions, such as phases and other parameters to monitor. In at least one embodiment, an irradiating raceway pond system 50 can include an embodiment of the invention for practicing the disclosed method by coupling an irradiation system 40A with a raceway pond platform to irradiate the microalgae culture (FIG. 5A). The irradiation system 40A with associated control system 50 can include, for example and without limitation, a housing 46 with a cooling system and electric installations that contain irradiation equipment 42, such as an X-ray tube, that can be placed in a irradiation casing 44, such as a lead box, with oil for heat dissipation (FIGS. 5B-5D). The irradiation system 40A can contain a controller 50 that can be connected to the sensor and used to control activation of irradiation system. The sensor can be, but is not limited to, an optical density probe or turbidity sensor. The sensor can provide a signal to the controller 50 to activate the irradiation equipment 42 at a predefined value of optical density or some other parameter of choice. The housing 46 can be placed on a framework with support 48 for mounting and stability. Additional protection from radiation can be provided by radiation shields 52 (“skirts”) coupled to the housing, such as a 0.5 mm lead layer (FIGS. 5B-5D). Radiation, such as X-rays, can be emitted through an irradiation window 54, such as a Be and AI window, into the culture 37 that flows by the irradiation system 40A. A beam angle θ and distance from the source can be optimized along with the beam to beam spacing S to allow coverage of large surface and maximal penetration of X-rays into the microalgae culture which is usually, but not limited to, 20-40 cm deep D in raceway ponds (FIG. 5E). In at least one embodiment, the irradiation system 40A can be transportable to irradiate other raceway ponds. In at least another embodiment, the irradiation system 40A can be fixedly installed on a raceway pond. The system requires an electric power supply. Other embodiments for an irradiating raceway pond system are possible and contemplated.

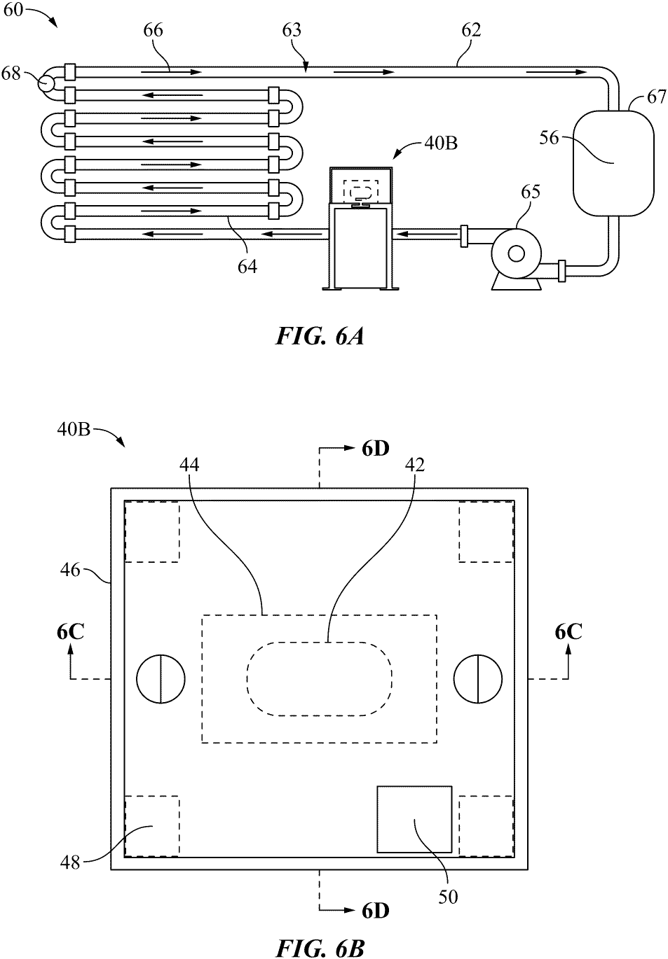

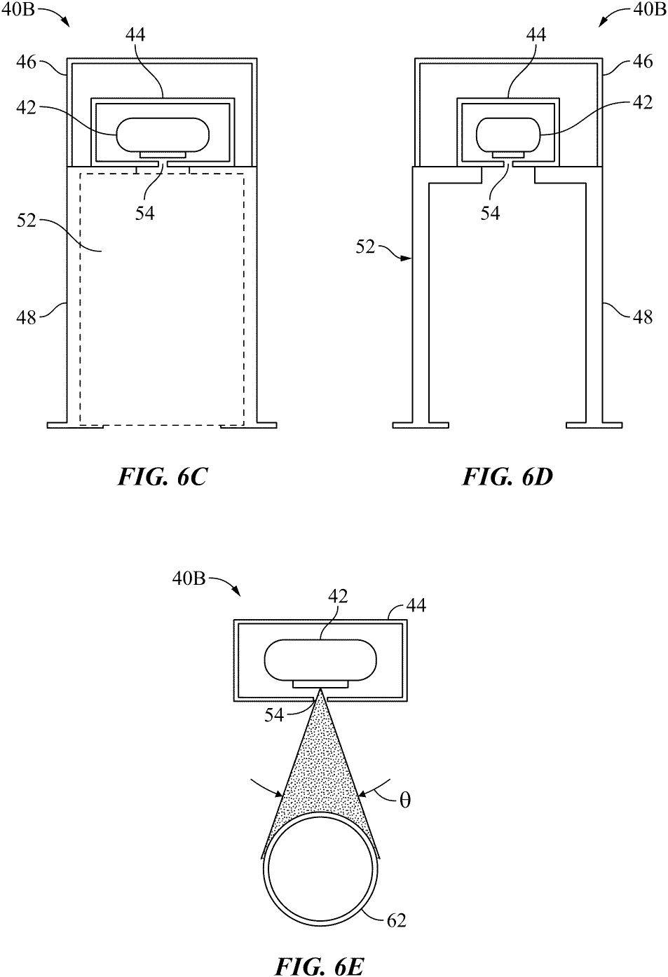

FIGS. 6A-6E are illustrative schematic diagrams of at least one embodiment of an irradiation system of the invention configured to apply an exemplary method to an existing microalgae cultivation platform of a photo-bioreactor under otherwise normal conditions. FIG. 6A is a side view schematic diagram of another example of an irradiation system that is mounted on a photo-bioreactor. FIG. 6B is an enlarged top view schematic diagram of the irradiation system of FIG. 6A. FIG. 6C is an enlarged side sectional view schematic diagram through a longitudinal section of the irradiation system of FIG. 6A. FIG. 6D is an enlarged side sectional view schematic diagram through a lateral section of the irradiation system of FIG. 6A. FIG. 6E is an enlarged side sectional view schematic diagram of irradiation beam coverage through a longitudinal section of the irradiation system of FIG. 6A.

A standard photo-bioreactor is known to one of ordinary skill in the art, and generally includes a conduit 62, such as a glass or plastic tube, to provide a flow circuit 66 for an aqueous growth substrate 56 that can flow through a solar collecting array 64, a motive force 65, such as a pump, to move a microalgae culture 63 in the conduit around the flow circuit, a feeding and degassing vessel 67, and a sensor 68 to sense culture conditions, such as phases and other parameters to monitor. In at least one embodiment, an irradiating photo-bioreactor system 60 can include an embodiment of the invention for practicing the method by, for example, coupling an irradiation system 40B to the photo-bioreactor in proximity to the conduit 62 (FIG. 6A). The irradiation system 40B with associated controller 50 can include, for example and without limitation, a housing 46 with a cooling system and electric installations that contain irradiation equipment 42, such as an X-ray tube, that can be placed in a irradiation casing 44, such as a lead box, with oil for heat dissipation (FIGS. 6B-6D). The irradiation system 40B can contain a controller 50 that can be connected to the sensor and used to control activation of irradiation system. The sensor can be, but is not limited to, an optical density probe or turbidity sensor. The sensor can provide a signal to the controller 50 to activate the irradiation equipment 42 at a predefined value of optical density or some other parameter of choice. The housing 46 can be placed on a framework with support 48 for mounting and stability. Additional protection from radiation can be provided by radiation shields 52 (“skirts”) coupled to the housing, such as a 0.5 mm lead layer (FIGS. 6C-6D). Radiation, such as X-rays, can be emitted through an irradiation window 54, such as a Be and AI window, into the culture 37 that flows by the irradiation system 40B. A beam angle θ and distance from the source can be optimized to allow coverage of a cross section of the tube (FIG. 6E). In at least one embodiment, the irradiation system 40B can be transportable to irradiate other photo-bioreactors. In at least another embodiment, the irradiation system 40B can be fixedly installed on a photo-bio-reactor. The system requires an electric power supply. Other embodiments for an irradiating photo-bioreactor system possible and contemplated.

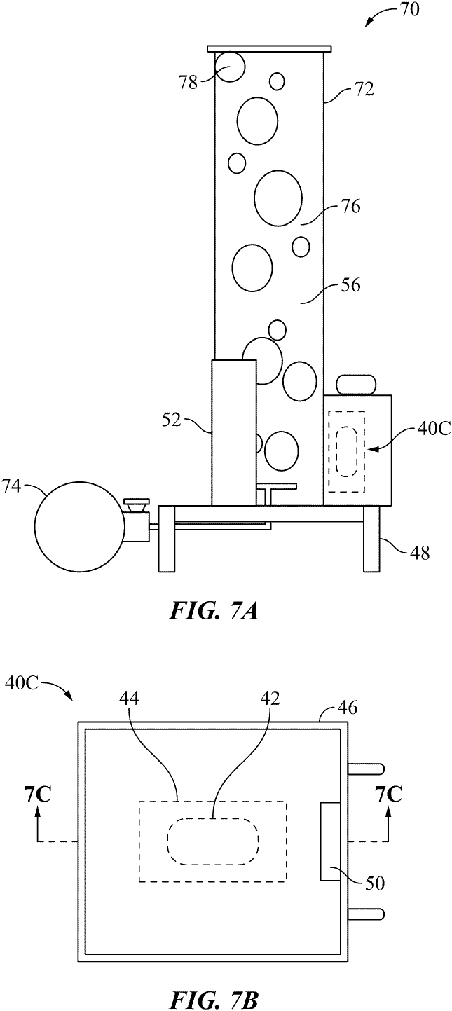

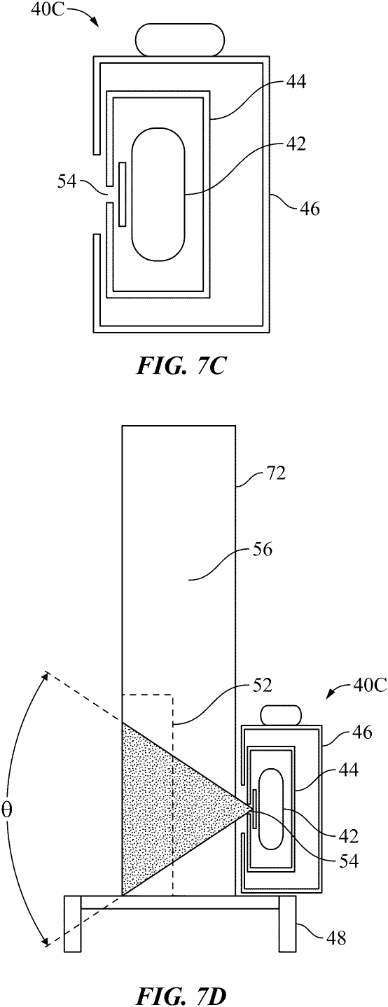

FIGS. 7A-7D are illustrative schematic diagrams of at least one embodiment of an irradiation system of the invention configured to apply an exemplary method to an existing microalgae cultivation platform of an airlift or bubble column bioreactor under otherwise normal conditions. FIG. 7A is a side view schematic diagram of another example of an irradiation system that is mounted on an airlift bioreactor. FIG. 7B is an enlarged top view schematic diagram of the irradiation system of FIG. 7A. FIG. 7C is an enlarged side sectional view schematic diagram through a longitudinal section of the irradiation system of FIG. 7A. FIG. 7D is an enlarged side sectional view schematic diagram of irradiation beam coverage through a longitudinal section of the irradiation system of FIG. 7A.

A standard airlift or bubble column -bioreactor is known to one of ordinary skill in the art, and generally includes a container 72 forming a bioreactor column, a motive force 74, such as an air pump, to inject gas forming gas bubbles into an aqueous growth substrate 56 for a microalgae culture 76 in the bioreactor column to agitate and mix the microalgae culture in the column or channel, and a sensor 78 to sense culture conditions, such as phases and other parameters to monitor. In at least one embodiment, an irradiating airlift or bubble column bioreactor 70 can include an embodiment of the invention for practicing the disclosed method by coupling an irradiation system 40C to irradiate the culture through a transparent glass or plastic surface in the container 72 (FIG. 7A). The irradiation system 40C with associated control system 50 can include, for example and without limitation, a system housing 46 with a cooling system and electric installations that contain irradiation equipment 42, such as an X-ray tube, that can be placed in a irradiation casing 44, such as a lead box, with oil for heat dissipation (FIGS. 7B-7C). The irradiation system 40C can contain a controller 50 that can be connected to the sensor and used to control activation of irradiation system. The sensor can be, but is not limited to, an optical density probe or turbidity sensor. The sensor can provide a signal to the controller 50 to activate the irradiation equipment 42 at a predefined value of optical density or some other parameter of choice. The housing 46 can be placed on a framework with support 48 for mounting and stability. Radiation, such as X-rays, can be emitted through an irradiation window 54, such as a Be and AI window, into the culture 76 in the airlift bioreactor column. A beam angle θ and distance from the source can be optimized to allow coverage of large surface and maximal penetration of X-rays into the microalgae culture (FIG. 7D). A radiation shield 52, such as with 0.5 mm lead layer, can provide protection from X-ray radiation that can be placed on the opposite side of the airlift or bubble column bioreactor compared to the irradiation system. In at least one embodiment, the irradiation system 40C can be transportable to irradiate other airlift bioreactors. In at least another embodiment, the irradiation system 40C can be fixedly installed on the airlift bioreactor. The system requires an electric power supply. Other embodiments for an irradiating airlift or bubble column bioreactor system are possible and contemplated.

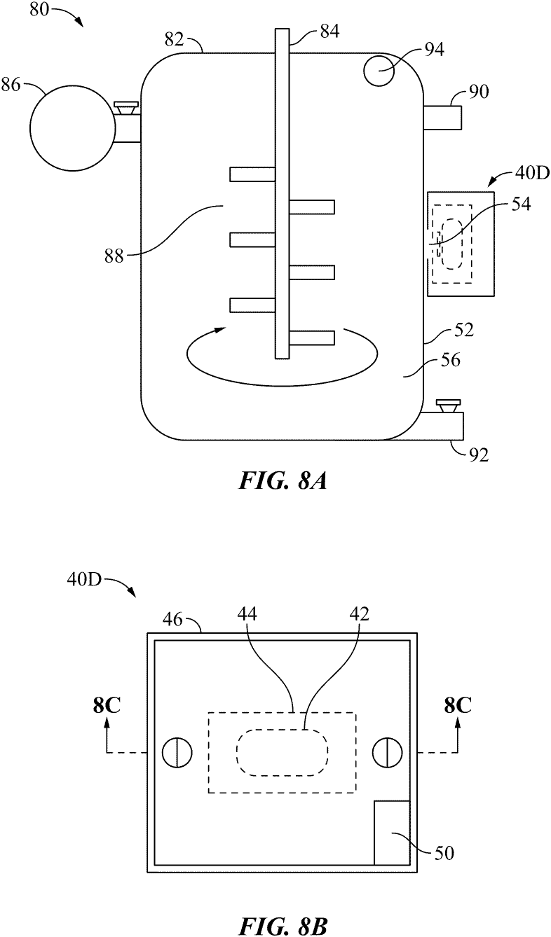

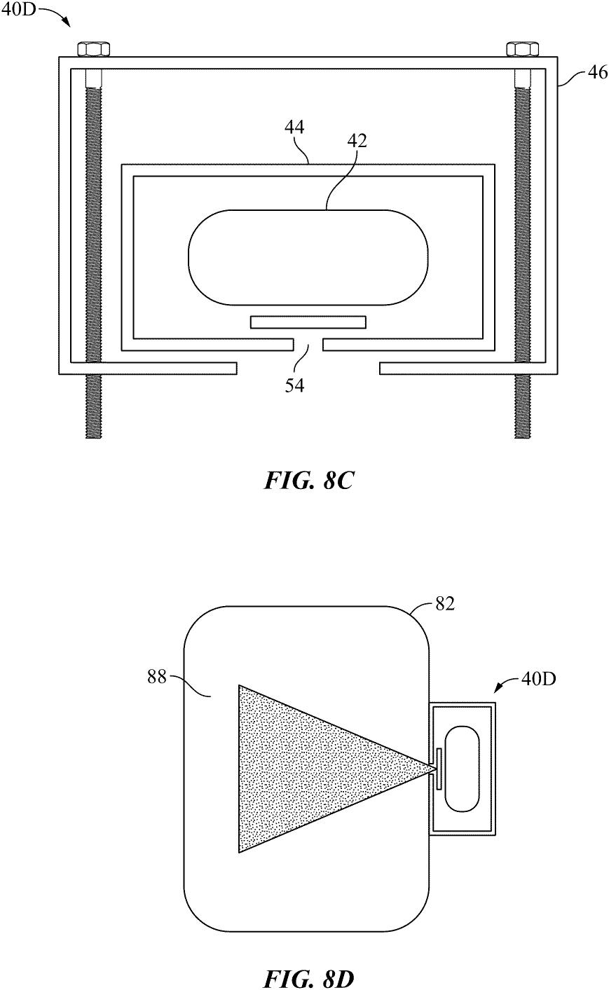

FIGS. 8A-8D are illustrative schematic diagrams of at least one embodiment of an irradiation system of the invention configured to apply an exemplary method to an existing microalgae cultivation platform of a fermentation bioreactor under otherwise normal conditions. FIG. 8A is a side view schematic diagram of another example of an irradiation system that is mounted on a wall of a fermentation bioreactor. FIG. 8B is an enlarged top view schematic diagram of the irradiation system of FIG. 8A. FIG. 8C is an enlarged side sectional view schematic diagram through a longitudinal section of the irradiation system of FIG. 8A. FIG. 8D is an enlarged side sectional view schematic diagram of irradiation beam coverage through a longitudinal section of the irradiation system of FIG. 8A.

A standard fermentation bioreactor is known to one of ordinary skill in the art, and generally includes a container 82 having an aqueous growth substrate 56 with an agitation system 84, a motive force 86, such as a pump, to inject a gas, such as air, into a microalgae culture 88 in the container, an air shaft 90 for ventilation, an effluent valve 92, and a sensor 94 to sense culture conditions, such as phases and other parameters to monitor. In at least one embodiment, an irradiating fermentation bioreactor 80 can include an embodiment of the invention for practicing the method by, for example, coupling an irradiation system 40D to the container 82. The irradiation system 40D with associated control system 50 can include, for example and without limitation, a housing 46 with a cooling system and electric installations that contain irradiation equipment 42, such as an X-ray tube, that can be placed in a irradiation casing 44, such as a lead box, with oil for heat dissipation (FIGS. 8B-8C). The irradiation system can be positioned to irradiate through a window 54, such as a Be and AI window, that allows radiation, such as X-rays, to reach the microalgae culture (FIGS. 8A and 8D). X-rays can irradiate only a part of the volume of fermentation bioreactor tank. A more uniform exposure of the microalgae culture to irradiation can be achieved by agitation of the culture (FIGS. 8A and 8D). A radiation shield 52, such as with 0.5 mm lead layer, can provide protection from X-ray radiation that can be placed on the container. The irradiation system 40D can contain a controller 50 that can be connected to the sensor and used to control activation of irradiation system. The sensor can be, but is not limited to, an optical density probe or turbidity sensor. The sensor can provide a signal to the controller 50 to activate the irradiation equipment 42 at a predefined value of optical density or some other parameter of choice. The housing 46 can be coupled to the container or can be placed on a framework with support for mounting and stability. A beam angle θ and distance from the source can be optimized to allow coverage of large surface and maximal penetration of X-rays into the microalgae culture (FIGS. 8). In at least one embodiment, the irradiation system 40D can be transportable to irradiate other fermentation bioreactors. In at least another embodiment, the irradiation system 40D can be fixedly installed on the container. The system requires an electric power supply. Other embodiments for an irradiating fermentation bioreactor system are possible and contemplated.

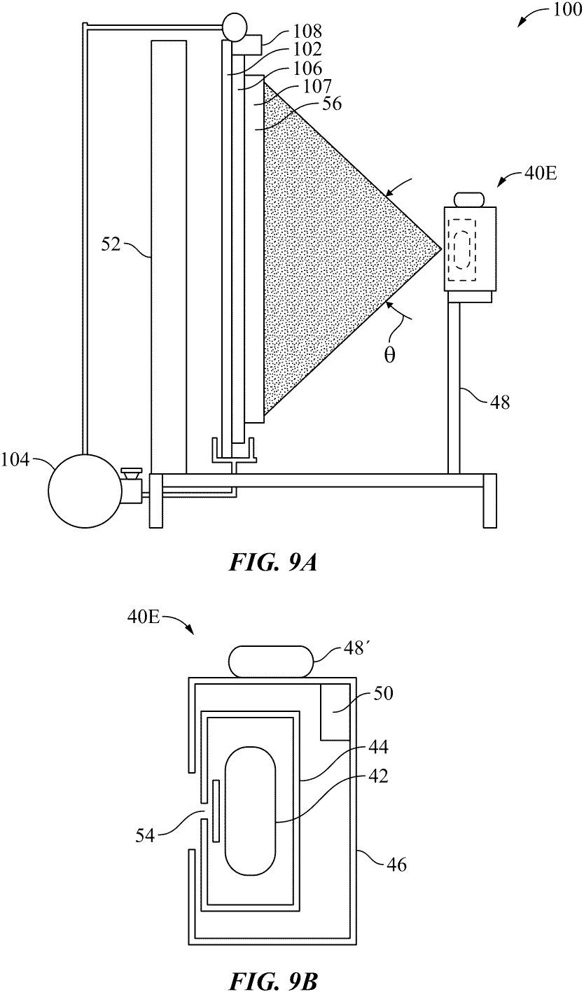

FIGS. 9A-9B are illustrative schematic diagrams of at least one embodiment of an irradiation system of the invention configured to apply an exemplary method to an existing microalgae cultivation platform of biofilm reactor under otherwise normal conditions. FIG. 9A is a side view schematic diagram of another example of an irradiation system that is mounted on biofilm reactor. FIG. 9B is an enlarged side sectional view schematic diagram through a longitudinal section of the irradiation system of FIG. 9A.

A standard biofilm reactor is known to one of ordinary skill in the art, and generally includes a source layer as a support surface 102, onto which a water and gas permeable/porous surface 106 is attached, through which water and gas may flow to provide a growth substrate 56, and microalgae 107 is retained on one side of the surface 106. Water and gas can be circulated by a motive force 104, such as a pump or injector, and a sensor 108 to sense conditions on the organism suitable for control of the pump and other equipment. In at least one embodiment, an irradiating biofilm reactor system 100 can include an embodiment of the invention for practicing the method by, for example, coupling an irradiation system 40E to the biofilm reactor in proximity support surface 102 with the microalgae (FIG. 9A). The irradiation system 40E with associated controller 50 can include, for example and without limitation, a housing 46 with a cooling system and electric installations that contain irradiation equipment 42, such as an X-ray tube, that can be placed in a irradiation casing 44, such as a lead box, with oil for heat dissipation (FIG. 9B). A support 48′, such as a handle, coupled with the housing can assist in mobility of the irradiation system. The irradiation system 40E can contain a controller 50 that can be connected to the sensor and used to control activation of the irradiation equipment 42 at a predefined value. The housing 46 can be placed on a support 48 for mounting and stability. Additional protection from radiation can be provided by radiation shields 52, such as a 0.5 mm lead layer, mounted on a distal side of the support surface 102 from the irradiation system 40E (FIG. 9A). Radiation, such as X-rays, can be emitted through an irradiation window 54, such as a Be and AI window, into the microalgae 107. A beam angle θ and distance from the source can be optimized to allow coverage of the microalgae (FIG. 9A). In at least one embodiment, the irradiation system 40E can be transportable to irradiate other biofilm reactors. In at least another embodiment, the irradiation system 40E can be fixedly installed on a biofilm reactor. The system requires an electric power supply. Other types of biofilm reactors, such as membrane aerated biofilm reactors, moving bed biofilm reactors, and others, can be adapted using the principles discussed herein and other embodiments of biofilm reactors are possible and contemplated.

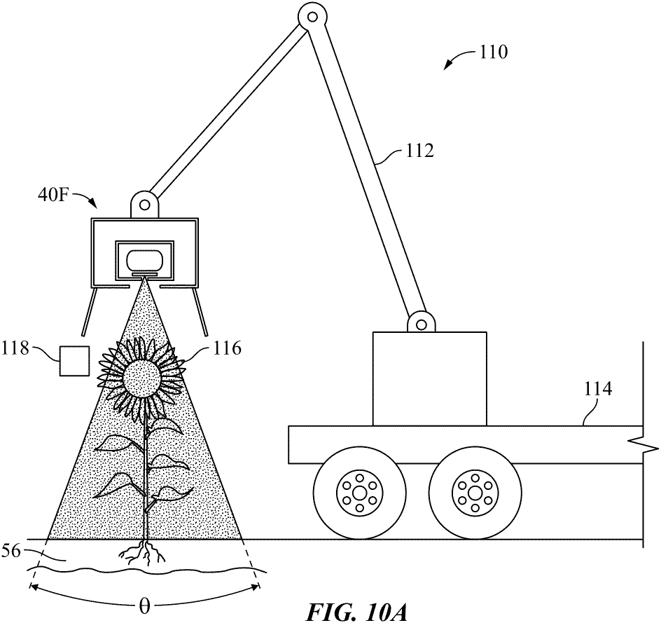

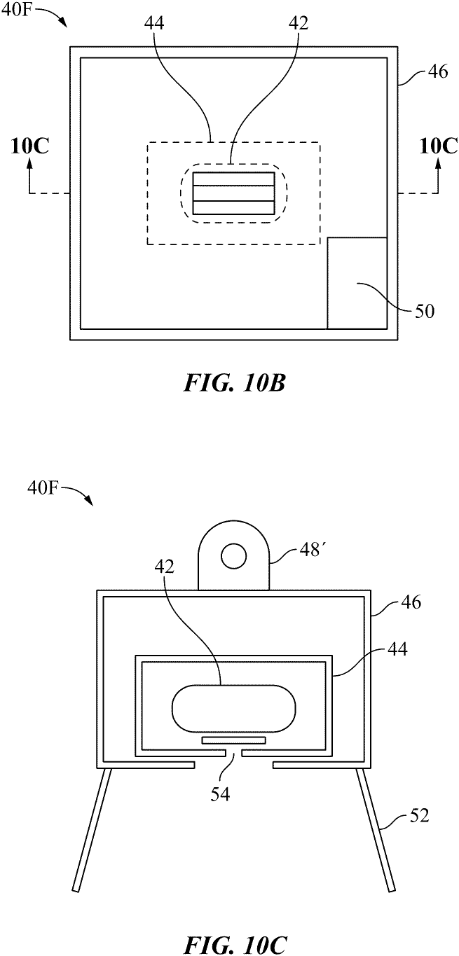

FIGS. 10A-10C are illustrative schematic diagrams of at least one embodiment of an irradiation system of the invention configured to apply an exemplary method to a culture of plants or other organisms with conserved metabolic pathways. FIG. 10A is a side view schematic diagram of another example of an irradiation system with multipurpose mobility for the treatment of organisms with conserved metabolic pathways relevant to lipid yield. FIG. 10B is an enlarged top view schematic diagram of the irradiation system of FIG. 10A. FIG. 10C is an enlarged side sectional view schematic diagram through a longitudinal section of the irradiation system of FIG. 10A.

The culture of microalgae and other organisms with conserved metabolic pathways relevant to lipid and other metabolite production are known to one of ordinary skill in the art. In at least one embodiment, a mobile platform with an irradiation system can be used for practicing the method. For example, a mobile irradiation system 110 can include using an irradiation system 40F that can be coupled with lifting equipment 112 using a mobile platform 114, such as trucks or other vehicles (FIG. 10A). In at least one embodiment, the lifting equipment 112 can include a crane or rail system for moving the irradiation system. The irradiation system 40F can be moved in proximity to such organism 116 supported by a growth substrate 56 for irradiation. The irradiation system 40F with associated control system 50 can include, for example and without limitation, a housing 46 with a cooling system and electric installations that contain irradiation equipment 42, such as an X-ray tube, that can be placed in a irradiation casing 44, such as a lead box, with oil for heat dissipation. (FIGS. 10B-10C) The irradiation system 40F can contain a controller 50 for operation of the irradiation equipment. In at least one embodiment, the irradiation system can be coupled to a sensor 118 used to control activation of irradiation system. The sensor can be suitable for sensing one or more parameters of the particular organism relevant for the irradiation activation. The sensor can provide a suitable signal (wired or wirelessly) to the controller 50 to activate the irradiation equipment 42. The housing 46 can be coupled to a support 48′, such as a swivel, that can be coupled to the lifting equipment 112. Additional protection from radiation can be provided by radiation shields 52 (“skirts”) coupled to the housing, such as a 0.5 mm lead layer (FIG. 10C). Radiation, such as X-rays, can be emitted through an irradiation window 54, such as a Be and AI window, onto the organism 116 by the irradiation system 40F. A beam angle θ and distance from the source can be optimized to allow coverage of large surface (FIG. 10A). The system can be easily transportable for irradiating different growing cultures and systems using the mobile platform. The system requires an electric power supply. Other embodiments on mobile irradiation systems are possible and contemplated.

Unless otherwise defined, all technical and scientific terms used herein have the same meaning as commonly understood by one of ordinary skill in the art to which this invention belongs. Suitable methods and materials are described herein, although methods and materials similar or equivalent to those described herein can be used in the practice or testing of the invention. All publications, patents, and other references mentioned herein are incorporated by reference in their entirety. In case of conflict, the present specification, including definitions herein, will control. In addition, the materials, methods, and examples are illustrative and not intended to be limiting.

Other and further embodiments utilizing one or more aspects of the invention described above can be devised without departing from the spirit of the Applicants’ invention. For example, the use of gamma irradiation instead of X-rays in the irradiation system, irradiation of organisms with conserved metabolic pathways, organisms that are grown on solid medium instead of liquid medium, irradiation with multiple doses, irradiation systems for alternative microalgae cultivation platforms or other plant and micro-organism cultivation systems, irradiation systems that are not mobile but integrated into bioreactors and other platforms, use of electromagnetic ionizing radiation to increase the yield of metabolites, various growth substrates, growth of microalgae and organisms with conserved metabolic pathways under conditions that are not normal according to the definition provided here, microalgae grown in other cultivation platforms, cultivation conditions, and culture types, other doses, rates and periods between irradiation and harvesting, other related and un-related microorganisms, along with other variations can occur in keeping within the scope of the claims.

The invention has been described in the context of preferred and other embodiments and not every embodiment of the invention has been described. Obvious modifications of the methods and systems include variations that a person with ordinary skill in the art would envision, given the teachings herein. The disclosed and undisclosed embodiments are not intended to limit or restrict the scope or applicability of the invention conceived of by the Applicant, but rather, in conformity with the patent laws. Applicants intend to protect fully all such modifications and improvements that come within the scope of the following claims.

What is claimed is:

1. A method of metabolic activation of an organism by irradiation, comprising:
irradiating the organism with at least one of X-ray and gamma radiation that produces hormesis in the organism, the organism being at least one of a microalgae strain and an organism with conserved metabolic pathways; and
metabolically priming the organism to produce a hormetic effect of at least one of a greater lipid yield, greater biomass yield, and greater metabolite yield under given conditions compared to untreated organisms of the same species when maintained under the same conditions.

2. The method of claim 1, wherein the hormetic effect occurs within microalgae cells in culture.

3. The method of claim 1, wherein the hormetic effect occurs with no significant decrease of biomass yield versus control on biomass yield.

4. The method of claim 2, wherein the culture is exposed to normal conditions for cultivation of a strain of the particular microalgae cells at a time of the irradiating and wherein the culture is maintained under the normal conditions after the irradiating for a period of time until harvesting.

5. The method of claim 2, wherein the culture is in a stationary phase of growth at a time of the irradiating.

6. The method of claim 5, wherein the irradiating has a hormetic effect of the greater lipid yield from the organism.

7. The method of claim 5, wherein the culture is exposed to normal conditions for cultivation of a strain of the particular microalgae strain at the time of the irradiating and wherein the culture is maintained under the normal conditions after the irradiating for a period of time until harvesting.

8. The method of claim 1, wherein the organism comprises at least one of:
microalgae strains comprising freshwater and marine algae, comprising Bacillariophyta (diatoms), Chlorophyta (green algae), Cyanobacteria (blue-green algae), Haptophyta (mostly marine algae including coccoliths), Phaeophyta (brown algae), Pyrrophycophyta (dinoflagellates), Rhodophyta (red algae); and
organisms with conserved metabolic pathways as the microalgae strain relevant to lipid yield, comprising Bigyra (including Thraustochytrids), Streptophyta (plants including mosses), Ascomycota (fungi including yeasts), Basidiomycota (fungi including mushrooms), bacteria, and archaea.

9. The system of claim 8, wherein
the organism is exposed to normal conditions that are recommended for cultivation at a time of the irradiating; and
the organism is maintained under the normal conditions after the irradiating for a period of time until harvesting.

10. The method of claim 1, wherein the hormetic effect comprises up-regulation of expression of lipid metabolism genes encoding enzymes that are involved in the biosynthesis of lipids in microalgae and organisms with conserved metabolic pathways relevant to lipid yield.

11. The method of claim 10, wherein the lipid metabolism genes comprise lipid metabolism genes for the production of fatty acids and triacylglycerols in lipid droplets.

12. The method of claim 10, wherein the organism comprises at least one of:
microalgae strains comprising freshwater and marine algae, comprising Bacillariophyta (diatoms), Chlorophyta (green algae), Cyanobacteria (blue-green algae), Haptophyta (mostly marine algae including coccoliths), Phaeophyta (brown algae), Pyrrophycophyta (dinoflagellates), Rhodophyta (red algae); and
organisms with conserved metabolic pathways as the microalgae strains relevant to lipid yield, comprising Bigyra (including Thraustochytrids), Streptophyta (plants including mosses), Ascomycota (fungi including yeasts), Basidiomycota (fungi including mushrooms), bacteria, and archaea.

13. The system of claim 12, wherein
the organism is exposed to normal conditions that are recommended for cultivation at a time of the irradiating; and
the organism is maintained under the normal conditions after the irradiating for a period of time until harvesting.

14. The method of claim 2, wherein the organism is in an exponential phase of growth at a time of the irradiating.

15. The method of claim 14, wherein the culture is exposed to normal conditions that are recommended for cultivation of a strain of the particular microalgae cells at the time of the irradiating and wherein the culture is maintained under the normal conditions after the irradiating for a period of time until harvesting.

16. The method of claim 14, wherein the irradiating has a hormetic effect of the greater biomass yield.

17. The method of claim 1, wherein the metabolite yield comprises omega-3 fatty acids.

18. The method of claim 17, wherein omega-3 fatty acids comprise alpha-linoleic acid.

19. A system for increasing performance of a culture of at least one of microalgae and organisms with conserved metabolic pathways by irradiation, comprising:
a cultivation platform comprising at least one of a raceway pond, a photo-bioreactor, an airlift bioreactor, a bubble column bioreactor, a fermentation bioreactor, and a biofilm reactor;
an irradiation system with a source of at least one of X-ray and gamma radiation, coupled to the cultivation platform; and
a controller configured to control activation of the source of radiation to irradiate the at least one of microalgae and organisms with conserved metabolic pathways at a predetermined phase of the culture for a period of time and rate of radiation and metabolically prime the at least one of microalgae and organisms with conserved metabolic pathways to produce a hormetic effect of at least one of a greater lipid yield, greater biomass yield, and greater metabolite yield of the culture under given conditions compared to an untreated culture of the same strain of the at least one of microalgae and organisms with conserved metabolic pathways when maintained under the same conditions.

20. The system of claim 19, further comprising a sensor configured to determine at least one parameter of at least one of the microalgae cells and microalgae cultivation platform and provide a signal to the controller to control the activation of the irradiation system.

21. The system of claim 19, wherein:
the culture is exposed to normal conditions that are recommended for cultivation of a strain of the particular microalgae cells at a time of the irradiating; and
the culture is maintained under the normal conditions after the irradiating for a period of time until harvesting.

22. A system for increasing performance of an organism with conserved metabolic pathways by irradiation, comprising:
a growth substrate for the organism;
an irradiation system with a source of at least one of X-ray or gamma radiation in operational proximity to the organism; and
a controller configured to control activation the irradiation system to irradiate the organism for a period of time and rate of radiation and metabolically prime the organism to produce a hormetic effect of at least one of a greater lipid yield, greater biomass yield, and greater metabolite yield under given conditions compared to an untreated organism of the same species when maintained under the same conditions.

23. The system of claim 22, wherein
the culture is exposed to normal conditions that are recommended for cultivation of the organism at a time of the irradiating; and
the organism is maintained under the normal conditions after the irradiating for a period of time until harvesting.
